(19) United States
(12) Patent Application Publication
(10) Pub. No.: US 2023/0390391 A1
(43) Pub. Date: Dec. 7, 2023

(54) BI-SPECIFIC CHIMERIC ANTIGEN RECEPTOR T CELLS TARGETING CD83 AND INTERLEUKIN 6 RECEPTOR

(71) Applicants: REGENTS OF THE UNIVERSITY OF MINNESOTA, Minneapolis, MN (US); H. LEE MOFFITT CANCER CENTER AND RESEARCH INSTITUTE, INC., Tampa, FL (US)

(72) Inventors: Brian BETTS, Minneapolis, MN (US); Marco DAVILA, Tampa, FL (US)

(21) Appl. No.: 17/759,271

(22) PCT Filed: Jan. 22, 2021
(86) PCT No.: PCT/US21/14730

§ 371 Date: Jul. 21, 2022

Related U.S. Application Data
(60) Provisional application No. 62/964,481, filed on Jan. 22, 2020.

(51) Int. Cl. A61K 39/00; C07K 16/28; A61P 37/06

(52) U.S. Cl. CPC A61K 39/4621; A61K 39/4631; C07K 16/2803; C07K 16/2866; A61K 39/4611; A61P 37/06; A61K 39/46433; A61K 2239/28; C07K 2317/622; A61K 2239/26

(57) ABSTRACT

A bi-specific genetically modified immune cell, comprising a first antigen binding moiety that is specific to CD83 and a second antigen binding moiety that is specific to interleukin 6 receptor (IL-6R). Also provided herein are uses of such bi-specific genetically modified immune cells for suppressing alloreactive donor cells in cell transplantation.

### CROSS REFERENCE TO RELATED APPLICATION

This application claims benefit under 35 U.S.C. § 119(e) of the U.S. provisional application No. 62/964,481 filed Jan. 22, 2020, the content of which is incorporated herein by reference is its entirety.

### BACKGROUND

Allogeneic hematopoietic cell transplantation (HCT) is an effective therapy for hematological malignancies but it is limited by acute graft-versus-host disease (GVHD). GVHD arises when donor T cells respond to genetically defined proteins on host cells, and is a key contributor to the high mortality associated with HCT. Dendritic cells (DC) play a major role in the allogeneic T cell stimulation causing GVHD. Donor DCs are the primary antigen presenting cell responsible for indirect presentation of alloantigens following transplantation, and this process commences almost immediately after transplantation. Current immunosuppressive measures to control GVHD target T cells but compromise post-transplant immunity in the patient.

### SUMMARY

The present disclosure is based, at least in part, on the development of bi-specific genetically modified immune cells capable of targeting both CD83 and IL-6 receptor (IL-6R) for use in suppressing alloreactive donor cells in a subject. The present disclosure reports that CD83 expression is significantly increased on conventional T cells (Tconv) in acute graft-versus host diseases (GvHD) and that IL-6Rα exclusively expresses on alloreactive CD83+ Tconv cells, leading to enhanced IL-6 signaling, which contributes to GvHD. Accordingly, the bi-specific genetically modified immune cells are expected to eliminate CD83+/IL-6R+ alloreactive donor cells in a subject receiving the donor cells, while preserving donor immunity (e.g., against cancer or infectious pathogens). Thus, the bi-specific genetically modified immune cells can protect transplant recipients from GvHD or solid organ allograft rejection without the risk for off-target lymphopenia. Additionally, the bi-specific genetically modified immune cells can prevent the rejection of third-party or “off-the-shelf” (e.g., HLA unmatched) cell therapy by host alloreactive cells (e.g., alloreactive T cells).

Accordingly, the present disclosure features, in some aspects, a bi-specific genetically modified immune cell (e.g., a T cell), comprising a first antigen binding moiety that is specific to CD83 and a second antigen binding moiety that is specific to interleukin 6 receptor (IL-6R).

In some embodiments, the bi-specific genetically modified immune cell (e.g., a dual CAR-T cell) may express a first chimeric antigen receptor (CAR) that comprises the first antigen binding moiety, and a second chimeric antigen receptor (CAR) that comprises the second antigen binding moiety. The first CAR may further comprise a first co-stimulatory signaling domain and a first intracellular signaling domain. The second CAR may further comprise a second co-stimulatory signaling domain and a second intracellular signaling domain.

In some embodiments, the bi-specific genetically modified immune cell (e.g., a tandem CAR T cell) may express a bi-specific chimeric antigen receptor (CAR), which comprises the first antigen binding moiety, the second antigen binding moiety, a co-stimulatory signaling domain, and an intracellular signaling domain.

In any of the bi-specific genetically modified immune cells disclosed herein, the first antigen binding moiety specific to CD83 is a single chain variable fragment (scFv) that binds CD83. In some embodiments, the scFv that binds CD83 comprises the same heavy chain complementary determining regions (CDRs) as a reference anti-CD83 antibody and/or the same light chain CDRs as the reference anti-CD83 antibody, and wherein the reference anti-CD83 antibody is GMB00, Clone 20D04, Clone 11G05, Clone 14C12, Clone 020B08, Clone 006G05, Clone 96G08, or Clone 95F04, In one example, the anti-CD83 scFv may comprise the same heavy chain and light chain CDRs as GMB00. In another example, the anti-CD83 scFv may comprise the same heavy chain and light chain CDRs as 96G08. In yet another example, anti-CD83 scFv may comprise the same heavy chain and light chain CDRs as 95F04.

In some examples, the anti-CD83 scFv may comprise the same heavy chain variable region (VH) as GMB01, GMB02, GMB03, GMB04, GMB05, or GMB06, and/or the same light chain variable region (VL) as GMB01, or GMB02. Alternatively, the anti-CD83 scFv may comprise the same VH and/or the same VL as the reference antibody Clone 20D04, Clone 11G05, Clone 14C12, Clone 020B08, Clone 006G05, Clone 96G08, or Clone 95F04. In specific examples, the anti-CD83 scFv may comprise an amino acid sequence selected from the group consisting of SEQ. ID. Nos.: 59-71.

Alternatively or in addition, the second antigen binding moiety specific to IL-6R in any of the bi-specific genetically modified immune cells disclosed herein may be a single chain variable fragment (scFv) that binds the IL-6R. In other embodiments, the second antigen binding moiety specific to IL-6R may be an IL-6R ligand, e.g., IL-6 or a fragment thereof that binds an IL-6 receptor. As used herein, an antigen binding moiety specific to IL-6R refers to any moiety that binds the protein complex of IL-6R, or a subunit thereof (e.g., IL-6Rα or GP130). In some instances, the IL-6R binding moiety binds IL-6Rα (a.k.a., CD126). In other instances, the IL-6R binding moiety binds GP130.

In some examples, the second antigen binding moiety specific to IL-6R is a single chain variable fragment (scFv), which may be derived from Tocilizumab, Sarilumab, and antibody clones ALX-0061, TZLS-501, and BCD-089 (e.g., having the same heavy chain and light chain CDRs or having the same VH and VL as any of the listed reference antibodies.)

In some embodiments, the first co-stimulatory signaling, the second co-stimulatory domain, and/or the co-stimulatory domain in the bispecific CAR can be a co-stimulatory signaling domain of CD27, CD28, 4-1BB, OX40, CD30, CD40, PD-1, ICOS, lymphocyte function-associated antigen-1 (LFA-1), CD2, CD7, LIGHT, NKG2C, or B7-H3. In some examples, the first co-stimulatory signaling domain, the second co-stimulatory signaling domain, and/or the co-stimulatory signaling domain in the bispecific CAR is a CD28 co-stimulatory signaling domain or a 4-1BB co-stimulatory signaling domain.

In some embodiments, the first intracellular signaling, the second intracellular signaling domain, and/or the intracellular signaling domain in the bispecific CAR can be a CD3ζ signaling domain.

Any of the CAR constructs disclosed herein, including an anti-CD83 CAR, an anti-IL-6R CAR, or a bispecific CAR disclosed herein, may further comprise a hinge domain, a transmembrane domain, or a combination thereof.

In some embodiments, the present disclosure features a bi-specific chimeric antigen receptor (CAR), wherein the bi-specific CAR comprises a first antigen binding moiety specific to CD83 and a second antigen binding moiety specific to IL-6R. The anti-CD83 moiety can be any of those disclosed herein and the anti-IL-6R moiety can be any of those disclosed herein.

In other aspects, provided herein is a method for suppressing alloreactive immune responses in a subject, the method comprising administering to a subject in need thereof an effective amount of the bi-specific genetically modified immune cells disclosed herein, thereby suppressing alloreactive donor cells in the subject. In some embodiments, the donor cells are bone marrow cells comprising alloreactive T-cells, dendritic cells, or a combination thereof. In some embodiments, the bi-specific genetically modified immune cells suppress alloreactive cells in the allogenic donor cells but preserve donor immunity against a target antigen, for example, a cancer antigens and/or an antigens from an infectious pathogens.

In some embodiments, the subject is a human patient in need of transplantation of the alloreactive donor cells. In some examples, the human patient may be at risk for GVHD. In other examples, the human patient may be at risk for rejection by the alloreactive cells.

In some embodiments, the subject has undergone or is undergoing a therapy comprising a checkpoint inhibitor. Examples include an anti-PD-1 antibody, an anti-PD-L1 antibody, or an anti-CTLA-4 antibody. As disclosed herein, CD83 is differentially expressed on allo-activated, human, conventional CD4+ T cells (Tconv), with minimal expression on regulatory T cells (Treg). To enhance the specificity of CAR T cell targeting of alloreactive T cells, a dual IL-6Rα/CD83 CAR T cell is disclosed herein to eliminate alloreactive T cells, yet preserve donor immunity against cancer and infectious pathogens.

Also provided herein are immune effector cells genetically modified to express at least two chimeric antigen receptor (CAR) polypeptides that can be used with adoptive cell transfer to suppress alloreactive cells, such as donor T cells. The first CAR polypeptide can contain in an ectodomain an antigen binding domain that can bind CD83 on cells (anti-CD83 agent). The second CAR polypeptide can contain in an ectodomain an antigen binding domain that can bind IL6 receptor on cells. In some embodiments, the immune effector cell can be selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell (Treg).

The IL6 receptor is a protein complex consisting of an IL-6 receptor subunit (IL6R) and interleukin 6 signal transducer Glycoprotein 130. Therefore, in some embodiments, the second CAR polypeptide can contain in an ectodomain an antigen binding domain that can bind IL6Rα (anti-IL6Rα agent). In some embodiments, the second CAR polypeptide can contain in an ectodomain an antigen binding domain that can bind GP130 (anti-GP130 agent). The antigen binding domain can also be a natural or synthetic ligand of IL6Rα, or a variant and/or fragment thereof capable of binding IL6Rα.

The antigen binding domain is in some embodiments an antibody fragment that specifically binds CD83, IL6Rα, or GP130. For example, the antigen binding domain can be a Fab or a single-chain variable fragment (scFv) of an antibody that specifically binds CD83, IL6Rα, or GP130.

The antigen binding domain is in some embodiments an aptamer that specifically binds CD83, IL6Rα, or GP130. For example, the antigen binding domain can be a peptide aptamer selected from a random sequence pool based on its ability to bind CD83, IL6Rα, or GP130.

In some embodiments, the anti-CD83 scFv can comprise a variable heavy (VH) domain having CDR1, CDR2 and CDR3 sequences and a variable light (VL) domain having CDR1, CDR2 and CDR3 sequences. For example, in some embodiments, the CDR1 sequence of the VH domain comprises the amino acid sequence GFSITTGGYWWT (SEQ ID NO:1), SDGIS (SEQ ID NO:7), or SNAMI (SEQ ID NO:13); CDR2 sequence of the VH domain comprises the amino acid sequence GYIFSSGNTNYNPSIKS (SEQ ID NO:2), IISSGGNTYYASWAKG (SEQ ID NO:8), or AMDSNSRTYYATWAKG (SEQ ID NO:14); CDR3 sequence of the VH domain comprises the amino acid sequence CARAYGKLGFDY (SEQ ID NO:3), VVGGTYSI (SEQ ID NO:9), or GDGGSSDYTEM (SEQ ID NO:15); CDR1 sequence of the VL comprises the amino acid sequence TLSSQHSTYTIG (SEQ ID NO:4), QSSQSVYNNDFLS (SEQ ID NO:10), or QSSQSVYGNNELS (SEQ ID NO:16); CDR2 sequence of the VL domain comprises the amino acid sequence VNSDGSHSKGD (SEQ ID NO:5), YASTLAS (SEQ ID NO:11), or QASSLAS (SEQ ID NO:17); and CDR3 sequence of the VL domain comprises the amino acid sequence GSSDSSGYV (SEQ ID NO:6), TGTYGNSAWYEDA (SEQ ID NO:12), or LGEYSISADNH (SEQ ID NO:18).

In some embodiments, the CDR1 sequence of the VH domain comprises the amino acid sequence GFSITTGGYWWT (SEQ ID NO:1), CDR2 sequence of the VH domain comprises the amino acid sequence GYIFSSGNTNYNPSIKS (SEQ ID NO:2), CDR3 sequence of the VH domain comprises the amino acid sequence CARAYGKLGFDY (SEQ ID NO:3), CDR1 sequence of the VL comprises the amino acid sequence TLSSQHSTYTIG (SEQ ID NO:4), CDR2 sequence of the VL domain comprises the amino acid sequence VNSDGSHSKGD (SEQ ID NO:5), and CDR3 sequence of the VL domain comprises the amino acid sequence GSSDSSGYV (SEQ ID NO:6).

In some embodiments, the CDR1 sequence of the VH domain comprises the amino acid sequence SDGIS (SEQ ID NO:7), CDR2 sequence of the VH domain comprises the amino acid sequence IISSGGNTYYASWAKG (SEQ ID NO:8), CDR3 sequence of the VH domain comprises the amino acid sequence VVGGTYSI (SEQ ID NO:9), CDR1 sequence of the VL comprises the amino acid sequence QSSQS VYNNDFLS (SEQ ID NO:10), CDR2 sequence of the VL domain comprises the amino acid sequence YASTLAS (SEQ ID NO:11), and CDR3 sequence of the VL domain comprises the amino acid sequence TGTYGNSAWYEDA (SEQ ID NO:12).

In some embodiments, the CDR1 sequence of the VH domain comprises the amino acid sequence SNAMI (SEQ ID NO:13), CDR2 sequence of the VH domain comprises the amino acid sequence AMDSNSRTYYATWAKG (SEQ ID NO:14), CDR3 sequence of the VH domain comprises the amino acid sequence GDGGSSDYTEM (SEQ ID NO:15), CDR1 sequence of the VL comprises the amino acid sequence QSSQSVYGNNELS (SEQ ID NO:16), CDR2 sequence of the VL domain comprises the amino acid sequence QASSLAS (SEQ ID NO:17), and CDR3 sequence of the VL domain comprises the amino acid sequence LGEYSISADNH (SEQ ID NO:18).

Any of the anti-CD83 binding moiety in the bi-specific genetically modified immune cells disclosed herein may have the same heavy chain and light chain complementary determining regions (CDRs) as any of the anti-CD83 antibodies listed in Table 1 (sequence table) provided below. In some examples, the anti-CD83 binding moiety may comprise the same heavy chain variable region (VH) and/or the same light chain variable region (VL) as the anti-CD83 antibodies provided in Table 1. In specific examples, the anti-CD83 binding moiety can be a single chain variable fragment (scFv) comprising an amino acid sequence of any one of SEQ ID NOs: 59-71.

As with other CARs, the disclosed polypeptides can also contain a transmembrane domain and an endodomain capable of activating an immune effector cell. For example, the endodomain can contain a signaling domain and one or more co-stimulatory signaling regions.

In some embodiments, the intracellular signaling domain is a CD3 zeta (CD3ζ) signaling domain. In some embodiments, the costimulatory signaling region comprises the cytoplasmic domain of CD28, 4-1BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1BB that enhance signaling.

In some embodiments, the CAR polypeptide contains an incomplete endodomain. For example, the CAR polypeptide can contain only an intracellular signaling domain or a co-stimulatory domain, but not both. In these embodiments, the immune effector cell is not activated unless it and a second CAR polypeptide (or endogenous T-cell receptor) that contains the missing domain both bind their respective antigens. Therefore, in some embodiments, the CAR polypeptide contains a CD3 zeta (CD3ζ) signaling domain but does not contain a costimulatory signaling region (CSR). In other embodiments, the CAR polypeptide contains the cytoplasmic domain of CD28, 4-1BB, or a combination thereof, but does not contain a CD3 zeta (CD3ζ) signaling domain (SD).

Also disclosed are isolated nucleic acid sequences encoding the disclosed CAR polypeptides, vectors comprising these isolated nucleic acids, and cells containing these vectors.

In some embodiments, the cell suppresses alloreactive donor cells, such as T cells, when the antigen binding domain of the CAR binds to CD83 and IL6 Receptor.

Also disclosed is a method of preventing GVHD in a subject that involves administering to the subject an effective amount of the disclosed immune effector cells. In some embodiments, the subject is receiving a tissue transplantation. In some embodiments, the tissue transplantation comprises a bone marrow transplantations. In some embodiments, the tissue transplantation comprises a solid organ transplant, including but not limited to, face transplant, abdominal wall transplant, limb transplant, upper extremity transplant, vascularized composite allograft, or whole tissue graft. In some embodiments, the subject has an autoimmune diseases, sepsis, rheumatological diseases, diabetes, and/or asthma. Also disclosed is a method of treating autoimmunity in a subject that involves administering to the subject an effective amount of the disclosed immune effector cells. Also disclosed is a method of preventing rejection of solid organ allografts and off-the-shelf CAR-T cells in a subject that involves administering to the subject an effective amount of the disclosed immune effector cells.

The details of one or more embodiments of the invention are set forth in the accompanying drawings and the description below. Other features, objects, and advantages of the invention will be apparent from the description and drawings, and from the claims.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1E include diagrams showing CD4+ T-cell IL-6 receptor activity is increased among alloHCT recipients before acute GVHD onset. FIG. 1A: a diagram illustrating IL-6 receptor and signaling apparatus. FIG. 1B: contour plots show expression of IL-6 receptor alpha on CD4+ T-cells in response to 3-day auto- or alloDC stimulation versus resting state (3 independent experiments). FIG. 1C: a diagram showing percentage of IL-6Rα+ CD4+ T-cells in resting T cells, T-cell+Autologous dendritic cells, and T-cells+allogenic dendritic cells. FIG. 1D: a plot showing CD4+ T-cell STAT3 phosphorylation at day +21 is highest among alloHCT recipients who develop severe acute GVHD by day +100 (n=5 healthy controls, n=10 grade 0-1, and n=8 grade II-IV). PBMCs were isolated from alloHCT recipients at day +21, then stimulated or not with 4000 IU/ml of IL-6. FIG. 1E: a graph shows cumulative incidence of acute GVHD by day +100 based on STAT3 activity of CD4+ T-cells at day +21. *P<0.05, **P=0.001-0.01, ***P=0.0001-0.001, ****P<0.0001.

FIGS. 2A-2E include diagrams showing that human CD83 CAR T cells prevent acute GVHD. FIG. 2A: a chart showing that human T cells were stimulated by allogeneic moDCs (DC:T cell ration 1:30). CD83 expression on activated Tconv (CD4+, CD127+, CD25+) or Treg (CD4+, CD127−, CD25+, Foxp3+) was measured at baseline, 4 hours, 8 hours, 24 hours, and 48 hours post stimulation. FIG. 2B: a chart showing that NSG mice received 25 ×10⁶ human PBMCs and were inoculated with low (1 ×10⁶) or high dose (10 ×10⁶) CD83 CAR or (1-10 ×10⁶) mock transduced T cells. The CARs were autologous to the PBMC donor. An additional control group of mice received PBMCs alone. Survival is shown. Pooled data from 3 independent experiments, up to 9 mice per experimental arm. FIGS. 2C and 2D: bar graphs showing the absolute numbers (mean±SEM) of CD4+, CD83+ and total CD4+ T cells in the mouse spleens at day +21, ANOVA. FIG. 2E: a bar graph shows CD83 expression on Tconv, Treg, and CD8+ T cells after stimulation with CD3/CD28 beads (Bead:T cell ratio 1:30). **P=0.001-0.01 and ****P<0.0001.

FIGS. 3A-3B include diagrams showing that IL-6Rα is co-expressed on alloreactive, CD83+ Tconv. FIG. 3A: a bar graph showing CD83/IL-6Rα co-expression on DC-allostimulated human T cells after 8 hours of stimulation. FIG. 3B: representative contour plots. n=4 independent experiments. ANOVA. ****=P<0.0001.

FIGS. 4A-4E include diagrams showing that IL-6 signaling is associated with GvHD development. FIG. 4A: a bar graph showing IL-6 stimulation of pSTAT3 expression. FIG. 4B: a bar graph showing increase of pSTAT3+CD4+ T cells along with GvHD development. FIGS. 4C and 4D: diagrams showing increase of pSTATs in Grade II GvHd versus no GvHD in the presence of IL-6. FIG. 4E: sensitivity v. specificity plot.

FIG. 5 is a bar graph showing that CD83+ conventional T cells (Tcon) are increased among alloHCT recipients with active acute GvHD. The proportion (±SEM) of CD83+, CD4+, CD4+ Tcon are significantly increased among alloHCT recipients with acute GVHD. N=4 healthy alloHCT patients and n=4 acute GVHD patients. Mann-Whitney. *P<0.05.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The present disclosure is based, at least in part, on the discovery that CD83 is differentially expressed on allo-activated, human, conventional CD4+ T cells (Tconv), with minimal expression on regulatory T cells (Treg). Moreover, CD83 positive Tconv cells also differentially expressed IL-6Rα. The expressions of CD83 and IL-6Rα were low in CD4+ non-Tconv cells, Treg cells, and CD8+ T cells, with or without dendrite cell mediated activation. To enhance the specificity of CAR T cell targeting of alloreactive T cells, a dual IL-6Rα/CD83 CAR T cell is disclosed herein to eliminate alloreactive T cells, yet preserve donor immunity against cancer and infectious pathogens. Furthermore, the dual IL-6Rα/CD83 CAR T cell would have low or no cytotoxicity against normal cells and tissues.

Described herein are bi-specific genetically modified immune cells targeting both CD83 and IL-6 receptor (IL-6R). In some embodiments, such bi-specific genetically modified immune cells may express at least one chimeric antigen receptor (CAR) specific to CD83 (anti-CD83 CAR) and at least one CAR specific to IL-6R (e.g., specific to IL-6α. In other embodiments, the bi-specific genetically modified immune cells may express a bi-specific CAR (e.g., a tandem CAR) capable of binding to both CD83 and IL-6R (e.g., IL-6a. Any of the anti-CD83 CAR, anti-IL-6R CAR, or bi-specific CAR to both CD83 and IL-6R, nucleic acids encoding such, genetically engineered T cells expressing such, therapeutic applications of such genetically engineered T cells, as well as methods for producing any of the bi-specific genetically engineered T cells and the T cells thus produced are also within the scope of the present disclosure.

### I. Chimeric Antigen Receptors (CARs)

Chimeric antigen receptor (CAR) T-cell therapy uses genetically-modified T cells to more specifically and efficiently target and kill undesired cells, such as cancer cells, in a mammal. After T cells have been collected from the blood, the cells are engineered to express CARs on their surface. In the present invention, alloreactive IL-6Rα+/CD83+ T cells are targeted to mitigate GVHD.

A chimeric antigen receptor (CAR), as used herein, refers to an artificial immune cell receptor that is engineered to recognize and bind to an antigen expressed by undesired cells, for example, disease cells such as cancer cells. CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). A CAR polypeptide can be introduced into immune cells such as T cells for surface expression to produce CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various designs of CARs, each of which contains different components. In some embodiments, CARs may join an antibody-derived scFv to the CD3zeta (CD3ζ) intracellular signaling domain of the T-cell receptor through hinge and transmembrane domains. In some embodiments, CARs incorporate an additional co-stimulatory domain, e.g., CD28, 4-1BB (41BB), or ICOS, to supply a costimulatory signal. In other embodiments, CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

In some instances, a CAR can be a fusion polypeptide comprising an extracellular antigen binding domain that recognizes a target antigen (e.g., a single chain variable fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain.

The disclosed CAR is generally made up of three domains: an ectodomain, a transmembrane domain, and an endodomain. The ectodomain comprises the extracellular antigen-binding domain(s) of a CAR.

The antigen recognition domain of the disclosed CAR is usually a scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

In this disclosure, the antigens targeted are CD83 and IL-6 receptor, e.g., the IL-6R complex or a subunit thereof, for example, IL-6Rα. Optionally, gp130 may be targeted. GP130 is associated with IL-6 receptor/IL-6 ligand interaction, activation, and cell signaling.

(a) Extracellular Antigen-Binding Domains

The extracellular antigen binding domain for the CARs disclosed herein is the region of any anti-CD83 CARs or any anti-IL-6R CARs (e.g., anti-IL-6Rα or anti-GP130 CARs) disclosed herein that is exposed to the extracellular fluid when the CAR is expressed on cell surface. In some embodiments, the antigen binding domain can be a single-chain variable fragment (scFv, which may include an antibody heavy chain variable region (VH) and an antibody light chain variable region (VL) (in either orientation). In some instances, the VH and VL fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized VH and/or VL domains. In other embodiments, the VH and/or VL domains of the scFv are fully human.

In some embodiments, the antigen-binding domains are the CD83 binding domains of a scFv or mAb, the IL-6Rα binding domains of a scFv or mAb, or the gp130 binding domains of a scFv or mAb. These include the scFv (heavy and light chains) and the CDR1, CDR2, and CDR3 regions in the Fv (heavy and light chains) of the scFv and mAb.

The ectodomain comprises the CD83, IL6Rα, or GP130 binding region and is responsible for antigen recognition. It also optionally contains a signal peptide (SP) so that the CAR can be glycosylated and anchored in the cell membrane of the immune effector cell.

Anti-CD83 Extracellular Antigen-Binding Domain

The extracellular antigen-binding domain in the CAR polypeptide disclosed herein is specific to CD83 (e.g., human CD83). In some examples, the extracellular antigen binding domain may comprise a scFv extracellular domain capable of binding to the CD83 antigen. The anti-CD83 scFv may be derived from antibody GBM00, GMM01, GMM02, GMM03, GMM04, GMM05, and GMM06. GBM00 is a mouse monoclonal antibody. GBM01-GBM06 are humanized antibodies derived for the mouse GBM00. Other anti-CD83 antibodies useful for constructing the anti-CD83 CAR include 20D04, 11G05, 14C12, 020B08, 006G05, 96G08, and See Table 1 below.

In some embodiments, an anti-CD83 scFv derived from GBM00 may comprise a heavy chain variable domain (VH) having the same heavy chain complementary determining regions (CDRs) as those in Antibody GBM00 and/or a light chain variable domain (VL) having the same light chain CDRs as those in GBM00. Two antibodies having the same VH and/or VL CDRs means that their CDRs are identical when determined by the same approach (e.g., the Kabat approach, the Chothia approach, the AbM approach, the Contact approach, or the IMGT approach as known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.bioinf.org.uk/abs/. The heavy chain and light chain CDRs of clone GBM00 are SEQ. ID. Nos: 1-6, and its VH and VL sequences are SEQ. ID. Nos.: 19 and 20.

In other embodiments, an anti-CD83 scFv derived from GBM00 may be a functional variant of GBM00. Such a functional variant is substantially similar to GBM00, both structurally and functionally. For example, a humanized form of the mouse GBM00. A functional variant comprises substantially the same VH and VL CDRs as GBM00. For example, it may comprise only up to 8 (e.g., 8, 7, 6, 5, 4, 3, 2, or 1) amino acid residue variations in the total CDR regions relative to those in GBM00 and binds the same epitope of CD83 with substantially similar affinity (e.g., having a KD value in the same order). In some instances, the functional variants may have the same heavy chain CDR3 as GBM00, and optionally the same light chain CDR3 as GBM00. Such an anti-CD83 scFv may comprise a VH fragment having CDR amino acid residue variations (e.g., up to 5) in only the heavy chain CDR1 and/or CDR2 as compared with the VH of GBM00. Alternatively or in addition, the anti-scFv antibody may further comprise a VL fragment having CDR amino acid residue variations (e.g., up to 5) in only the light chain CDR1 and/or CDR2 as compared with the VL of GBM00. In some examples, the amino acid residue variations can be conservative amino acid residue substitutions.

In some embodiments, the anti-CD83 scFv can comprise a variable heavy (VH) domain having CDR1, CDR2 and CDR3 sequences and a variable light (VL) domain having CDR1, CDR2 and CDR3 sequences. For example, in some embodiments, the CDR1 sequence of the VH domain comprises the amino acid sequence GFSITTGGYWWT (SEQ ID NO:1), SDGIS (SEQ ID NO:7), or SNAMI (SEQ ID NO:13); CDR2 sequence of the VH domain comprises the amino acid sequence GYIFSSGNTNYNPSIKS (SEQ ID NO:2), IISSGGNTYYASWAKG (SEQ ID NO:8), or AMDSNSRTYYATWAKG (SEQ ID NO:14); CDR3 sequence of the VH domain comprises the amino acid sequence CARAYGKLGFDY (SEQ ID NO:3), VVGGTYSI (SEQ ID NO:9), or GDGGSSDYTEM (SEQ ID NO:15); CDR1 sequence of the VL comprises the amino acid sequence TLSSQHSTYTIG (SEQ ID NO:4), QSSQSVYNNDFLS (SEQ ID NO:10), or QSSQSVYGNNELS (SEQ ID NO:16); CDR2 sequence of the VL domain comprises the amino acid sequence VNSDGSHSKGD (SEQ ID NO:5), YASTLAS (SEQ ID NO:11), or QASSLAS (SEQ ID NO:17); and CDR3 sequence of the VL domain comprises the amino acid sequence GSSDSSGYV (SEQ ID NO:6), TGTYGNSAWYEDA (SEQ ID NO:12), or LGEYSISADNH (SEQ ID NO:18).

In some embodiments, the anti-CD83 extracellular antigen-binding domain comprises a VH domain and a VL domain. The CDR1 sequence of the VH domain comprises the amino acid sequence GFSITTGGYWWT (SEQ ID NO:1), CDR2 sequence of the VH domain comprises the amino acid sequence GYIFSSGNTNYNPSIKS (SEQ ID NO:2), CDR3 sequence of the VH domain comprises the amino acid sequence CARAYGKLGFDY (SEQ ID NO:3), CDR1 sequence of the VL comprises the amino acid sequence TLSSQHSTYTIG (SEQ ID NO:4), CDR2 sequence of the VL domain comprises the amino acid sequence VNSDGSHSKGD (SEQ ID NO:5), and CDR3 sequence of the VL domain comprises the amino acid sequence GSSDSSGYV (SEQ ID NO:6).

In another embodiment, the CDR1 sequence of the VH domain comprises the amino acid sequence SDGIS (SEQ ID NO:7), CDR2 sequence of the VH domain comprises the amino acid sequence IISSGGNTYYASWAKG (SEQ ID NO:8), CDR3 sequence of the VH domain comprises the amino acid sequence VVGGTYSI (SEQ ID NO:9), CDR1 sequence of the VL comprises the amino acid sequence QSSQS VYNNDFLS (SEQ ID NO:10), CDR2 sequence of the VL domain comprises the amino acid sequence YASTLAS (SEQ ID NO:11), and CDR3 sequence of the VL domain comprises the amino acid sequence TGTYGNSAWYEDA (SEQ ID NO:12).

In further embodiment, the CDR1 sequence of the VH domain comprises the amino acid sequence SNAMI (SEQ ID NO:13), CDR2 sequence of the VH domain comprises the amino acid sequence AMDSNSRTYYATWAKG (SEQ ID NO:14), CDR3 sequence of the VH domain comprises the amino acid sequence GDGGSSDYTEM (SEQ ID NO:15), CDR1 sequence of the VL comprises the amino acid sequence QSSQSVYGNNELS (SEQ ID NO:16), CDR2 sequence of the VL domain comprises the amino acid sequence QASSLAS (SEQ ID NO:17), and CDR3 sequence of the VL domain comprises the amino acid sequence LGEYSISADNH (SEQ ID NO:18).

In some embodiments, the anti-CD83 binding moiety may comprise a VH domain may comprise an amino acid sequence of any one of SEQ ID NOs: 19 and 48-53. Alternatively or in addition, the anti-CD83 binding moiety may comprise a VL domain that may comprise an amino acid sequence of any one of SEQ ID NOs: 20, 54, and 55. In the anti-CD83 binding moieties disclosed herein, any of the VH domains of SEQ ID NOs: 19 and 48-53 may pair with any of the VL domains of SEQ ID NOs: 20, 54, and 55.

In some embodiments, the anti-CD83 extracellular antigen-binding domain contain humanized sequences in the VH or VL or both VH and VL of the disclosed antibodies. In some embodiments, the anti-CD83 extracellular antigen-binding domain comprise the humanized VH chains of SEQ. ID. Nos.: 48-53 which are derived for clones GMB01-GMB06 respectively. Six exemplary humanized VH chains are described in SEQ. ID. Nos.: 48-53 (from GMB01-GMB06 respectively) and two exemplary humanized VL chains are described in SEQ. ID. Nos.: 54-55 (from GMB01-GMB02 respectively).

In some embodiments, anti-CD83 extracellular antigen-binding domain may have the different combinations of VH and VL chains derived from antibody GBM00, and the humanized antibodies GMB01-GMB06. The VH and VL are linked together in a single polypeptide to form the extracellular antigen-binding domain. The heavy and light chains are preferably separated by a linker. Suitable linkers for scFv antibodies are known in the art. In some embodiments, the linker comprises the amino acid sequence GGGGSGGGGSGGGGS (SEQ ID NO: 56).

In some embodiments, the anti-CD83 scFv may comprise humanized VH and VL chains derived from GBM00 (e.g., any of the humanized GMB01-GMB06 VH chain in combination with any of the humanized GMB01-02 VL chains. In some examples, the anti-CD83 scFv may be in the format of, from N-terminus to C-terminus, VH-linker-VL. In other examples, the anti-CD83 scFv may be in the format of, from N-terminus to C-terminus, VL-linker-VH.

Other combinations include:
a VH fragment of SEQ ID NO: 19 and a VL fragment of SEQ ID NO: 54;
a VH fragment of SEQ ID NO: 19 and a VL fragment of SEQ ID NO: 55;
a VH fragment of SEQ ID NO: 48 and a VL fragment of SEQ ID NO: 20;
a VH fragment of SEQ ID NO: 48 and a VL fragment of SEQ ID NO: 54;
a VH fragment of SEQ ID NO: 48 and a VL fragment of SEQ ID NO: 55;
a VH fragment of SEQ ID NO: 49 and a VL fragment of SEQ ID NO: 20;
a VH fragment of SEQ ID NO: 49 and a VL fragment of SEQ ID NO: 54;
a VH fragment of SEQ ID NO: 49 and a VL fragment of SEQ ID NO: 55;
a VH fragment of SEQ ID NO: 50 and a VL fragment of SEQ ID NO: 20;
a VH fragment of SEQ ID NO: 50 and a VL fragment of SEQ ID NO: 54;
a VH fragment of SEQ ID NO: 50 and a VL fragment of SEQ ID NO: 55;
a VH fragment of SEQ ID NO: 51 and a VL fragment of SEQ ID NO: 20;
a VH fragment of SEQ ID NO: 51 and a VL fragment of SEQ ID NO: 54;
a VH fragment of SEQ ID NO: 51 and a VL fragment of SEQ ID NO: 55;
a VH fragment of SEQ ID NO: 52 and a VL fragment of SEQ ID NO: 20;
a VH fragment of SEQ ID NO: 52 and a VL fragment of SEQ ID NO: 54;
a VH fragment of SEQ ID NO: 52 and a VL fragment of SEQ ID NO: 55;
a VH fragment of SEQ ID NO: 53 and a VL fragment of SEQ ID NO: 20;
a VH fragment of SEQ ID NO: 53 and a VL fragment of SEQ ID NO: 54; and
a VH fragment of SEQ ID NO: 53 and a VL fragment of SEQ ID NO: 55.

In specific examples, the anti-CD83 scFv may comprise the amino acid sequence of SEQ ID NO: 59-71.

Anti-IL6Rα Extracellular Antigen-Binding Domain

The extracellular antigen-binding domain in the CAR polypeptide disclosed herein is specific to IL-6 receptor (IL6R), for example, IL6Rα (e.g., human IL6Rα) or GP130 (e.g., human GP130). In some examples, the extracellular antigen binding domain may comprise a scFv extracellular domain capable of binding to the IL-6 receptor antigen. The anti-IL-6Rα scFv may be derived from antibodies ALX-0061 or BCD-089. Structure information of these anti-IL-6R antibodies are known in the art.

In some embodiments, an anti-IL6Rα scFv derived from ALX-0061 or BCD-089 may comprise a heavy chain variable domain (VH) having the same heavy chain complementary determining regions (CDRs) as those in ALX-0061 or BCD-089 and/or a light chain variable domain (VL) having the same light chain CDRs as those in ALX-0061 or BCD-089.

In other embodiments, an anti-IL6Rα scFv derived from ALX-0061 or BCD-089 may be a functional variant of ALX-0061 or BCD-089. Such a functional variant is substantially similar to ALX-0061 or BCD-089, both structurally and functionally. A functional variant comprises substantially the same VH and VL CDRs as ALX-0061 or BCD-089.

In some embodiments, an anti-IL6Rα scFv derived from the antibody clones ALX-0061, TZLS-501 or BCD-089 may comprise a heavy chain variable domain (VH) having the same heavy chain complementary determining regions (CDRs) as those from tocilizumab or sarilumab; and/or a light chain variable domain (VL) having the same light chain CDRs as those in derived from tocilizumab and sarilumab as disclosed. Tocilizumab and sarilumab are humanized anti-IL-6 receptor antibodies that are known in the art (U.S. Pat. Nos. 8,562,991, 7,582,298, and 10,759,862, the contents are hereby incorporated by reference in their entireties).

In other embodiments, an anti-IL6Rα scFv derived from tocilizumab and sarilumab may be a functional variant of tocilizumab and sarilumab. Such a functional variant is substantially similar to tocilizumab and sarilumab, both structurally and functionally. A functional variant comprises substantially the same VH and VL CDRs as tocilizumab and sarilumab.

(b) Transmembrane-Hinge Domain

The anti-CD83, anti-IL-6R CAR polypeptides (e.g., anti-IL6Rα or anti-gp130 CAR polypeptide), or the bi-specific anti-IL-6Rα/CD83 CAR disclosed herein may contain a transmembrane domain (TD), which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

The TD connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In one specific example, the transmembrane domain in the anti-CD83 CAR, anti-IL-6R CAR, or dual IL-6Rα/CD83 CAR is a CD8c transmembrane domain.

In other embodiments, the transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp.

Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety and the transmembrane domain. The hinge sequence can be any suitable sequence derived or obtained from any suitable molecule. In some embodiments, for example, the hinge sequence is derived from a CD8a molecule or a CD28 molecule.

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

(c) Endodomain of the CAR

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” (ISD) of a T cell receptor (TCR) and optional co-receptors (e.g., co-stimulatory receptors). While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

In some embodiments, the endodomain of the CARs described herein comprises a cytoplasmic signaling domain (SD) and a co-stimulatory signaling domain (CSR). For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. In some examples, the CSR is derived from 4-1BB. In other examples, the CSR is derived from CD28. Optionally, the CAR may contain an additional co-stimulatory signaling domain.

In some embodiments, the endodomain of the CARs described herein comprises a SD and more than one CSR. There may be two CSRs or three CSRs. The multiple CSRs may be derived from the same source or a separate source. For example, a first CSR that is derived from CD3ζ and a second CSR that is derived from CD28.

In some embodiments, the CAR polypeptide contains an incomplete endodomain. For example, the CAR polypeptide can contain only an intracellular signaling domain or a co-stimulatory domain, but not both. In these embodiments, the immune effector cell is not activated unless it and a second CAR polypeptide (or endogenous T-cell receptor) that contains the missing domain both bind their respective antigens. Therefore, in some embodiments, the CAR polypeptide contains a CD3 zeta (CD3ζ) signaling domain but does not contain a co-stimulatory signaling region (CSR). In some embodiments, the CAR polypeptide described herein contains the cytoplasmic CSR of CD28, 4-1BB, or a combination thereof, but does not contain a CD3 zeta (CD3ζ) signaling domain (SD). In other embodiments, the CAR polypeptide described herein contains a CD3 zeta (CD3ζ) signaling domain (SD) but does not contain any cytoplasmic CSR.

Intracellular Signaling Domain (ISD)

Intracellular signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRIγ (FCERIG).

A “signaling domain (SD)” activates a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

Any of the anti-CD83 CAR, anti-IL-6R CAR, or dual IL-6Rα/CD83 CAR constructs disclosed herein contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three ITAMs, which transmit an activation signal to the T cell after the T cell is engaged with a cognate antigen. In many cases, CD3ζ provides a primary T cell activation signal but not a fully competent activation signal, which requires a co-stimulatory signaling. In some examples, the anti-CD83 CAR, anti-IL-6R CAR, or dual IL-6Rα/CD83 CAR construct disclosed herein comprise a CD3ζ cytoplasmic signaling domain.

In some embodiments, the anti-CD83 CAR, anti-IL-6R CAR, or dual IL-6Rα/CD83 CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule, for example, a CD28 co-stimulatory signaling domain. In other examples, the CAR disclosed herein comprises a 4-1BB co-stimulatory molecule, for example, a 4-1BB co-stimulatory signaling domain.

In specific examples, an anti-CD83 CAR, anti-IL-6R CAR, or dual IL-6Rα/CD83 CAR disclosed herein may include a CD3ζ signaling domain and a CD28 co-stimulatory domain.

It should be understood that methods described herein encompasses more than one suitable CAR that can be used to produce genetically engineered T cells expressing the CAR, for example, those known in the art or disclosed herein.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

Co-Stimulatory Signaling Domain (CSR)

The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD123, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the endodomain contains an ISD or a CSR, but not both. In these embodiments, an immune effector cell expressing such a CAR is only activated if another CAR (or a T-cell receptor) containing the missing domain also binds its respective antigen.

(d) Anti-CD83 CAR

In some embodiments, the anti-CD83 CAR is single chain variable fragment (scFv) antibody. The affinity/specificity of an anti-CD83 scFv is driven in large part by specific sequences within complementarity determining regions (CDRs) in the heavy (VH) and light (VL) chain. Each VH and VL sequence will have three CDRs (CDR1, CDR2, CDR3).

In some embodiments, the anti-CD83 CAR is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

The anti-CD83 CAR may comprise (a) an anti-CD83 extracellular antigen binding e.g., an anti-CD83 scFv derived from antibody clones GMB00, GMB01, GMB02, GMB03, GMB04, GMB05, GMB06, 20D04, 11G05, 14C12, 020B08, 006G05, 96G08, and 95F04; (b) a co-stimulatory signaling domain such as those disclosed herein; and (c) a cytoplasmic signaling domain such as those disclosed herein. The anti-CD83 CAR may further comprise a hinge domain and a transmembrane domain located at the C-terminal of the extracellular antigen binding domain.

In other embodiments, the anti-CD83 CAR may comprise (a) an extracellular binding domain that binds CD83, (b) a transmembrane domain, optionally a hinge domain, and (c) an intracellular SD or a CSR, optionally both an intracellular SD and a CSR.

In some aspects, provided herein are anti-CD83 CAR, nucleic acids encoding such, and host cells expressing such.

(e) Anti-IL-6 CAR

In some examples, the anti-IL-6R CAR may comprise (a) an extracellular binding domain which can be any of the anti-IL-6R binding moieties. The anti-IL-6R binding moiety disclosed herein may be capable of binding to the IL-6R complex comprising IL-6Rα and GP130. Alternatively, the anti-IL-6R binding moiety may bind to one subunit of the IL-6R receptor, for example, capable of binding to IL-6Rα or capable of binding to GP130. In some examples, the anti-IL-6R CAR disclosed herein binds IL-6Rα (e.g., human IL-6Rα). The extracellular binding domain of the anti-IL-6R CAR disclosed herein may comprise an anti-IL-6α scFv derived from monoclonal antibody clones TZLS-501, ALX-0061, BCD-089, or tocilizumab and sarilumab, which are known in the art; (b) a co-stimulatory signaling domain such as those disclosed herein; and (c) a cytoplasmic signaling domain such as those disclosed herein. The anti-IL-6a CAR may further comprise a hinge domain and a transmembrane domain located at the C-terminal of the extracellular antigen binding domain.

In other embodiments, the anti-IL-6R CAR may comprise (a) an extracellular binding domain that binds IL-6R, (b) a transmembrane domain, optionally a hinge domain, and (c) an intracellular SD or a CSR, optionally both an intracellular SD and a CSR.

In some aspects, provided herein are anti-IL-6α CAR, nucleic acids encoding such, and host cells expressing such.

(f) Multi-Chain Dual Anti-CD83/Anti-IL-6R CAR

In some embodiments, the CAR described herein is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In other instances, the anti-CD83/IL-6Rα bispecific CAR may be a multiple-chain (e.g., 2-chain) molecule. The anti-CD83 binding domain and the anti-IL-6Rα binding domain may be located on separate polypeptides. These separate polypeptides comprise a transmembrane domain and an endodomain for each polypeptide.

In some instances, the CAR described herein may comprise one of the co-stimulatory signaling domain and the intracellular signaling domain but not both. Such a CAR is designed to work only in conjunction with another CAR, which may bind a different antigen. The second CAR (or endogenous T-cell) provides the missing signal if it is activated. For example, if the bi-specific CAR contains an SD but not a CSR, then the immune effector cell containing this CAR is only activated if another CAR (or T-cell) containing a CSR binds its respective antigen. Likewise, if the bi-specific CAR contains a CSR but not a SD, then the immune effector cell containing this CAR is only activated if another CAR (or T-cell) containing an SD binds its respective antigen. For example, the anti-CD83 binding domain containing polypeptide has a SD in its endodomain and the anti-IL-6Rα binding domain containing polypeptide has a CSR in its endodomain.

(g) Bispecific Anti-CD83/Anti-IL-6R CAR

In some aspects, provided herein is anti-CD83/IL-6Rα bispecific CAR comprising an anti-CD83 binding domain (e.g., an anti-CD83 scFv such as those disclosed herein), an anti-IL-6Rα binding domain (e.g., scFv derived from TZLS-501, ALX-0061, BCD-089, tocilizumab and sarilumab as disclosed), one or more intracellular signaling domains such as co-stimulatory signaling domains and cytoplasmic signaling domains, and optionally a hinge domain and a transmembrane domain as disclosed herein. In some instances, the anti-CD83/IL-6Rα bispecific CAR may be a single polypeptide comprising both the anti-CD83 binding domain and the anti-IL-6Rα binding domain.

In some embodiments, the anti-CD83/IL-6Rα bispecific CAR disclosed herein may comprise an anti-CD83 binding domain (e.g., scFv) derived from GMB00 (e.g., SEQ ID NOs: 59-71 provided in Table 1 below) and an anti-IL-6Rα binding domain (e.g., scFv) derived from an anti-IL-6R antibody known in the art, such as tocilizumab, sarilumab, ALX-0061, TZLS-501 and BCD-089 as disclosed herein.

(h) Additional CAR Format

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored

CAR, Self-destruct CAR, Conditional CAR, Marked CAR, TenCAR, Dual CAR, or sCAR.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets. In some embodiments, a TanCAR can be a bi-specific CAR, in which the two scFv moieties bind to two different antigens (in this case, CD83 and IL-6R).

A dual CAR T cell expresses two separate CARs with different ligand binding targets; one CAR includes only the CD3ζ domain and the other CAR includes only the co-stimulatory domain(s). Dual CAR T cell activation requires co-expression of both targets.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

Any of the CAR format disclosed herein may be applied for constructing the anti-CD83 CAR, the anti-IL-6R CAR, or the bi-specific CAR disclosed herein.

### II. Nucleic Acids and Vectors

Also disclosed are polynucleotides and polynucleotide vectors encoding the disclosed CARs that allow expression of the CARs in the disclosed immune effector cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### III. Bispecific Genetically Engineered Immune Effector Cells

Also disclosed are immune effector cells that are engineered to express the disclosed CARs. These are sometimes referred to as CAR-T cells when the immune cells are T cells. The immune effector cells used for the genetic engineering are preferably obtained from the subject to be treated (i.e. are autologous).

In some embodiments, the genetically engineered immune effector cells expressing an anti-CD83/IL-6Rα bispecific CAR as described herein.

The CAR may be a single polypeptide that can target both CD83 and IL-6Rα. Alternatively, the CAR may formed from two separate polypeptides as described herein, one polypeptide targets CD83 and the second polypeptide targets IL-6Rα.

In one embodiment, the genetically engineered immune effector cells two CARs, one targeting CD83 and the other targeting IL-6Rα.

Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, TH9, or TFH, which secrete different cytokines to facilitate a different type of immune response.

Cytotoxic T cells (TC cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described—naturally occurring Treg cells and adaptive Treg cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8+ T lymphocytes. In some embodiments, the T cells comprise γδ T cells, which possess a distinct T-cell receptor (TCR) having one γ chain and one δ chain instead of α and β chains.

Natural-killer (NK) cells are CD56+CD3− large granular lymphocytes that can kill virally infected and transformed cells, and constitute a critical cellular subset of the innate immune system (Godfrey J, et al. Leuk Lymphoma 2012 53:1666-1676). Unlike cytotoxic CD8+ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

In some embodiments, the bi-specific genetically engineered immune cells may express an anti-CD83 CAR and an anti-IL-6R CAR, or a bispecific CAR that contains one of a co-stimulatory domain and an intracellular signaling domain but not both. For example, the bi-specific genetically engineered immune cells may express a CAR targeting CD83 and/or IL-6R comprising either a co-stimulatory domain or an intracellular signaling domain, but not both; nor comprising no signaling domain at all. Such bi-specific genetically engineered immune cells may co-express a CAR that comprises the missing signaling domain. The anti-CD83 CAR, anti-IL-6R, or the bispecific CAR would be active only in conjugation with the other CAR that provides the missing signaling domain. In some examples, an anti-CD83 CAR may supply the missing signaling domain for an anti-IL-6R CAR. In other examples, an anti-IL-6R CAR may supply the missing signaling domain for an anti-CD83 CAR.

### IV. Therapeutic Methods

Engineered immune effector cells expressing the disclosed CARs suppress alloreactive donor cells, such as T-cells, and prevent GVHD. Therefore, the disclosed bi-specific genetically modified immune cells, which target both CD83 and IL-6R, can be administered to any subject at risk for GVHD (e.g., a human patient who has undergone allogeneic cell transplantation, is underdoing allogeneic cell transplantation, or will be subject to allogeneic cell transplantation). Accordingly, in some embodiments, this disclosure provides a composition comprising a population of any of the bi-specific genetically engineered immune cells targeting both CD83 and IL-6R (e.g., expressing both anti-CD83 CAR and anti-IL-6R CAR or the bispecific CAR as described). The composition may be used to treat GVHD or to prevent GVHD in a subject at risk of developing GVHD (for example, in a transplant recipient). In some embodiments, the composition is a pharmaceutical composition.

In some embodiments, the subject receives a bone marrow transplant and the disclosed CAR-modified immune effector cells suppress alloreactivity of donor T-cells or dendritic cells. The disclosed CAR-modified immune effector cells may be administered either alone, or as a pharmaceutical composition in combination with diluents and/or with other components such as IL-2, IL-15, or other cytokines or cell populations.

In some embodiments, the disclosed CAR-modified immune effector cells are administered in combination with ER stress blockade (compounds to target the IRE-1/XBP-1 pathway (e.g., B-I09). In some embodiments, the disclosed CAR-modified immune effector cells are administered in combination with a JAK2 inhibitor, a STATS inhibitor, an Aurora kinase inhibitor, an mTOR inhibitor, or any combination thereof.

Briefly, pharmaceutical compositions may comprise a population of CAR-expressing immune effector cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate for treatment of GVHD. The quantity and frequency of administration will be determined by such factors as the condition of the patient, and the severity of the patient's disease, although appropriate dosages may be determined by clinical trials.

In some embodiments, provided herein is a population of CAR-expressing immune effector cells as described herein for use in the treatment or prevention of GVHD or organ transplant rejection. The CAR-modified immune effector cells may be allogenous or autologous to the recipient of the cells.

In some embodiments, the disclosed CAR-expressing immune effector cells are administered to target and eliminate CD83+/IL-6R+ T cells in a subject. The subject may be a recipient of a transplantation, e.g., organ transplant or bone marrow transplant. The subject may have developed GVHD or may be at risk of developing GVHD. For example, the recipient had experienced GVHD from a prior transplant.

When a “therapeutic amount” is indicated, the precise amount of the compositions of the present invention to be administered can be determined by a physician with consideration of individual differences in age, weight, extent of transplantation, and condition of the patient (subject). It can generally be stated that a pharmaceutical composition comprising the engineered immune effector cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by, i.v., injection. The compositions may also be injected directly into a site of transplantation.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a first antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tissue, and is advantageous in situations where certain normal tissues may express very low levels of an antigen that would lead to off target effects with a standard CAR (Morgan 2010). A conditional CAR-T cell expresses an extracellular antigen binding domain connected to an intracellular costimulatory domain and a separate, intracellular co-stimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

### V. Kits for Therapeutic Applications

The present disclosure also provides kits for use of the genetically engineered immune cells (e.g., T lymphocytes, NK cells, or macrophages) expressing anti-CD83/IL-6Rα bispecific CAR described herein. Such kits may include one or more containers comprising the genetically engineered immune cells, which may be formulated in a pharmaceutical composition further comprising a pharmaceutically acceptable carrier.

In some embodiments, the kit described herein comprises genetically engineered immune cells, which may be expanded in vitro. The immune cells may express any of the CAR disclosed herein, for example, any of the anti-CD83/IL-6Rα bispecific CARs as disclosed.

In some embodiments, the kit can additionally comprise instructions for use in any of the methods described herein. The included instructions may comprise a description of administration of the genetically engineered immune cells disclosed herein to achieve the intended activity, e.g., eliminating T cells expressing CD83, IL-6Rα, or both, in a subject. The kit may further comprise a description of selecting a subject suitable for treatment based on identifying whether the subject is in need of the treatment.

The instructions relating to the use of the genetically engineered immune cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the genetically engineered immune cells are used for treating GVHD in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### Definitions

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “ligand” refer to a polypeptide comprising an IL6 fragment that binds the IL-6R.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e g immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M−1 (e.g., 10⁶ M−1, 10⁷ M−1, 10⁸ M−1, 10⁹ M−1, 10¹⁰ M−1, 10¹¹ M−1, and 10¹² M−1 or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

A number of embodiments of the invention have been described. Nevertheless, it will be understood that various modifications may be made without departing from the spirit and scope of the invention. Accordingly, other embodiments are within the scope of the following claims.

In additional to the exemplary embodiments disclosed herein, the present disclosure provides additional exemplary embodiments below.

### Additional Exemplary Embodiments

In some embodiments, provided herein is an immune effector cell genetically modified to express a first chimeric antigen receptor (CAR) polypeptide and a second CAR, wherein the first CAR comprises a CD83 binding domain, a transmembrane domain, an intracellular signaling domain, and a co-stimulatory signaling region, and wherein the second CAR comprises an IL6 Receptor binding domain, a transmembrane domain, an intracellular signaling domain, and a co-stimulatory signaling region.

In some examples, the IL6 Receptor binding domain in the immune effector cell disclosed in this section is a single-chain variable fragment (scFv) of an antibody that specifically binds IL6Rα. In some examples, the IL6 Receptor binding domain is a single-chain variable fragment (scFv) of an antibody that specifically binds GP130. In other examples, the IL6 Receptor binding domain is an IL6Rα ligand.

In some examples, the CD83 binding domain in any of the immune effector cells disclosed in this section is a single-chain variable fragment (scFv) of an antibody that specifically binds CD83. In some instances, the anti-CD83 scFv comprises a variable heavy (VH) domain having CDR1, CDR2 and CDR3 sequences and a variable light (VL) domain having CDR1, CDR2 and CDR3 sequences, wherein the CDR1 sequence of the VH domain comprises the amino acid sequence SEQ ID NO:1, SEQ ID NO:7, or SEQ ID NO:13; the CDR2 sequence of the VH domain comprises the amino acid sequence SEQ ID NO:2, SEQ ID NO:8, or SEQ ID NO:14; the CDR3 sequence of the VH domain comprises the amino acid sequence SEQ ID NO:3, SEQ ID NO:9, or SEQ ID NO:15; the CDR1 sequence of the VL comprises the amino acid sequence SEQ ID NO:4, SEQ ID NO:10, or SEQ ID NO:16; the CDR2 sequence of the VL domain comprises the amino acid sequence SEQ ID NO:5, SEQ ID NO:11, or SEQ ID NO:17; and the CDR3 sequence of the VL domain comprises the amino acid sequence SEQ ID NO:6, SEQ ID NO:12, or SEQ ID NO:18.

In some instances, the anti-CD83 scFv VH domain in any of the immune effector cells disclosed in this section comprises the amino acid sequence SEQ ID NO:19, SEQ ID NO:48, SEQ ID NO:49, SEQ ID NO:50, SEQ ID NO:51, SEQ ID NO:52, or SEQ ID NO:53. Alternatively or in addition, the anti-CD83 scFv VL domain comprises the amino acid sequence SEQ ID NO:20, SEQ ID NO:54, or SEQ ID NO:55. In specific examples, the anti-CD83 scFv comprises the amino acid sequence SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, or SEQ ID NO:71.

In any of the immune effector cells disclosed in this section, the costimulatory signaling region comprises the cytoplasmic domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. Alternatively or in addition, the intracellular signaling domain comprises a CD3 zeta (CD3ζ) signaling domain.

Any of the immune effector cells disclosed in this section can be a cell selected from the group consisting of an αβT cell, γδT cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, or any combination thereof.

Any of the immune effector cells disclosed in this section may suppress alloreactive donor cells when the first CAR binds to CD83 and the second CAR binds to IL6 Receptor on the donor cells.

In other embodiments, provided herein is a method of suppressing alloreactive donor cells in a subject receiving transplant donor cells, the method comprising administering to the subject an effective amount of the immune effector cell disclosed in this section, thereby suppressing alloreactive donor cells in the subject. In some instances, the donor cells are bone marrow cells comprising alloreactive T-cells, dendritic cells, or a combination thereof. In some instances, the method comprises further administration of a checkpoint inhibitor, which may comprise an anti-PD-1 antibody, anti-PD-L1 antibody, anti-CTLA-4 antibody, or a combination thereof.

### EXAMPLES

### Example 1: Dual IL-6Rα/CD83-Targeted CAR T Cells for Mediating Immune Tolerance

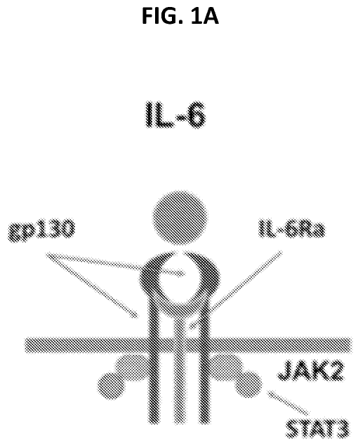

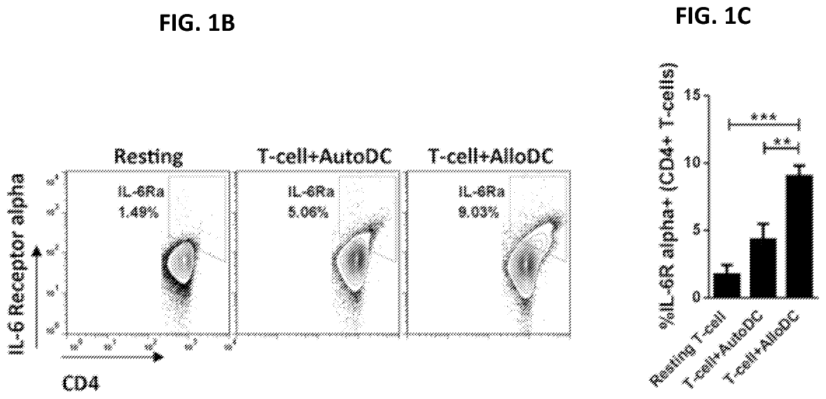

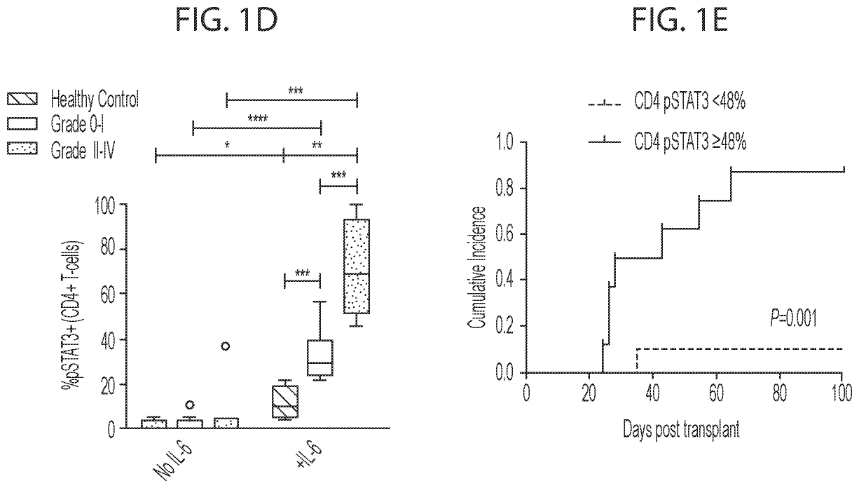

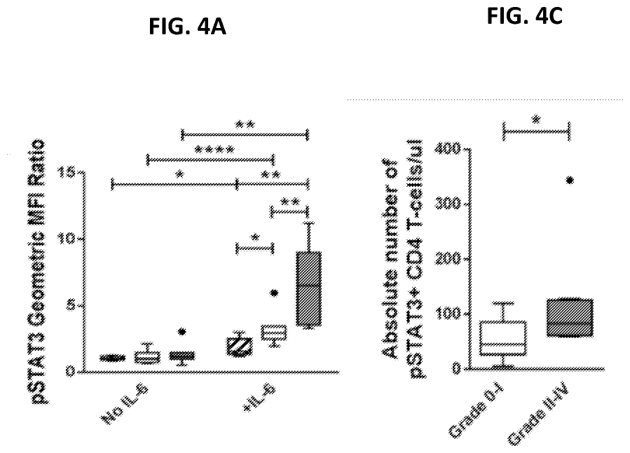

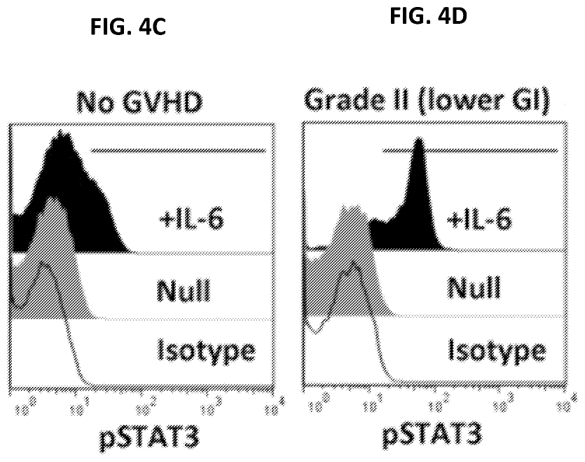

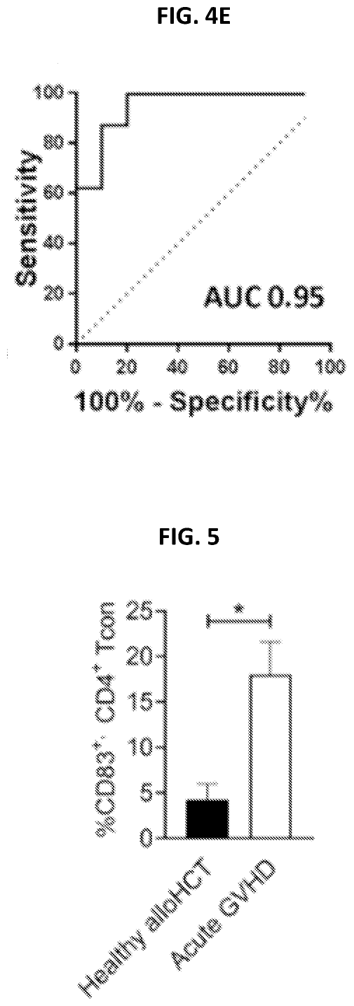

IL-6 receptor activation induces T-cell alloreactivity and promotes graft-versus-host disease (GVHD). GVHD is a leading cause of non-relapse mortality after allogeneic hematopoietic cell transplantation (alloHCT). GVHD occurs when donor T cells are intolerant of the host. IL-6 signaling polarizes Th1 and Th17 cells that are effectors in GVHD, and impairs Tregs that modulate GVHD. IL-6 facilitates STATS phosphorylation via JAK2 kinase (FIG. 1A). (Betts B C, et al. Journal of leukocyte biology. 2015; 97(4):807-19). It was observed that CD4+ T-cell JAK2/STATS activity is increased by IL-6 in alloHCT recipients who later develop GVHD (FIGS. 1B-1E). (Betts B C, et al. Journal of leukocyte biology. 2015; 97(4):807-19). See also FIGS. 4A-4E. These data show that IL-6 signals contribute to GVHD but prevention is incomplete when targeting the IL-6 pathway alone, as recently demonstrated by a negative randomized clinical trial testing the efficacy of tocilizumab (anti-IL-6 receptor mAb) against placebo (Kennedy G A, et al. ASH Annual Meeting 2019). Moreover, broadly inhibiting JAK1 and JAK2 downstream of the IL-6 receptor with ruxolitinib ameliorates the symptoms of GVHD (Spoerl S, et al. Blood. 2014; 123(24):3832-42), but its broad immune effects increase the risk for opportunistic infections by impairing natural killer cells and cytotoxic T lymphocytes (Betts B C, et al. Proc Natl Acad Sci USA. 2018; 115(7):1582-7; Curran S A, et al. Cancer immunology research. 2016).

Human CD83 CAR T cells prevent acute GVHD. For over 30 years, strategies to prevent

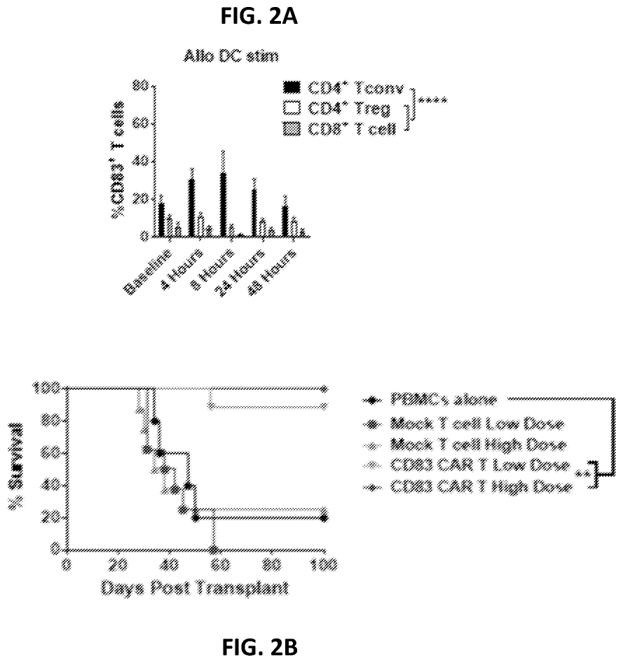

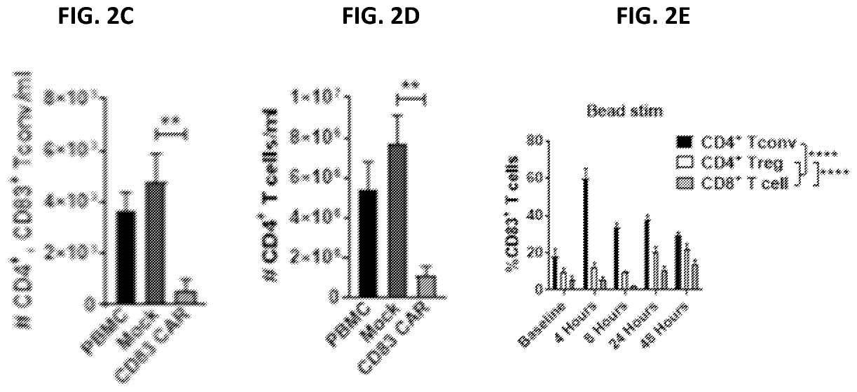

GVHD have included broadly suppressive pharmacologic agents, such as calcineurin-inhibitors. However, calcineurin-inhibitors provide incomplete protection from GVHD and impair the graft-versus-leukemia (GVL) effect, which is a fundamental benefit of allo-HCT. Distinct from pharmacologic immune suppression, we have developed a novel, donor-derived, human CD83 chimeric antigen receptor (CAR) T cell for GVHD prevention that uses a 4-1BB co-stimulatory domain and CD3ζ activation domain (Li G, et al. JCI Insight. 2018; 3(18); Li G, et al. Methods Mol Biol. 2017; 1514:111-8). CD83 is differentially expressed on allo-activated, human, conventional CD4+ T cells (Tconv), with minimal expression on regulatory T cells (Treg) (FIG. 2A) (Shrestha B, et al. ASH Annual Meeting. 2019). In a human T cell mediated xenogeneic GVHD model, CD83 CAR T cells were shown to provide lasting protection from alloreactive T cells (FIG. 2B) (Shrestha B, et al. ASH Annual Meeting. 2019). Similarly, CD83 CAR T cells can effectively treat acute GVHD and directly kill AML blasts without impairing normal hematopoiesis. The CD83 CAR T cell further influences T cell differentiation by abrogating pathogenic Th1 differentiation and significantly increasing the ratio of Treg to Tconv (Shrestha B, et al. ASH Annual Meeting. 2019). While the CD83 CAR T cell eliminates CD83+ effectors of GVHD (FIG. 2C) (Shrestha B, et al. ASH Annual Meeting. 2019), a reduction in total CD4+ T cells overall was also observed (FIG. 2D) (Shrestha B, et al. ASH Annual Meeting. 2019). Interestingly, supraphysiologic T cell activation with CD3/CD28 crosslinking beads results in CD83 expression on T cells (FIG. 2E) (Shrestha B, et al. ASH Annual Meeting. 2019). This suggests that strong T cell stimulation could induce CD83 expression on nonalloreactive T cells, rendering them susceptible to the CD83 CAR T cell. As such a dual IL-6Rα/CD83-targeted CAR T cell is expected to prevent and treat GVHD by provided exquisite specificity against alloreactive T cells, without broader effects on activated, but non-allosensitized T cells. Further, the need for the concurrent expression of IL-6Rα and CD83 would protect dendritic cells (DC), as immature DCs express IL-6Rα but not CD83 and mature DCs conversely express CD83 over IL-6Rα.

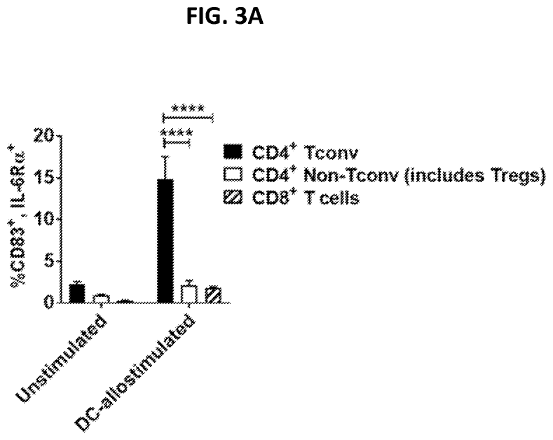

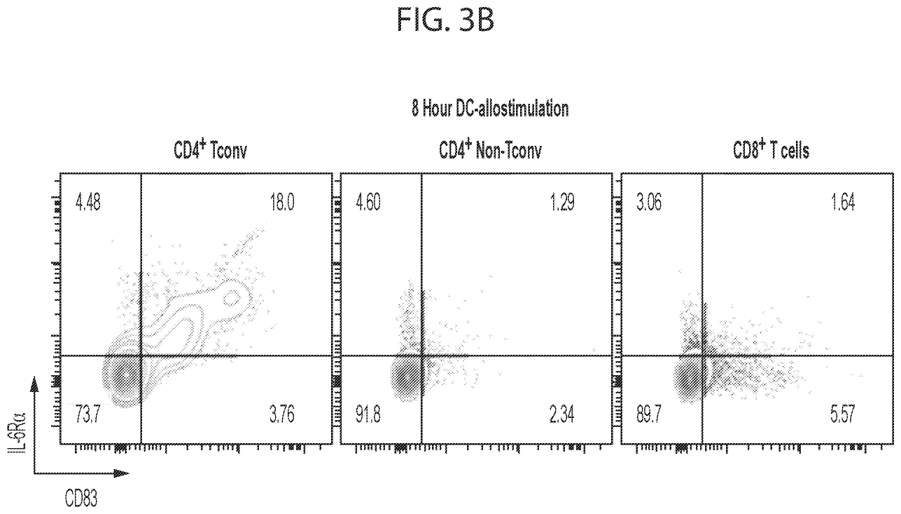

Alloreactive concurrently express IL-6Rα and CD83. To enhance the specificity of CAR T cell targeting of alloreactive T cells, a dual IL-6Rα/CD83 CAR T cell was developed to eliminate alloreactive T cells, yet preserve donor immunity against cancer and infectious pathogens. IL-6Rα expression is exclusive to the alloreactive CD83+ Tconv, compared to non-Tconv, CD8+ T cells, or resting T cells in general (FIGS. 3A and 3B). Thus, dual IL-6Rα/CD83 CAR T cell can protect transplant recipients from GVHD without any risk for off-target lymphopenia.

Further, FIG. 5 provides direct evidence showing that CD83+ conventional T cells are increased among alloHCT recipients with active acute GVHD. This is consistent with data from human T cells in vivo from xenogeneic GvHD experiments and in vitro studies.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

TABLE 1
Antibody Sequences

| Name | | Sequences | SEQ ID |
|---|---|---|---|
| GBM00 | VH | GFSITTGGYWWT (SEQ ID NO: 1) | 1 |
| | CDR1 | | |
| | VH | GYIFSSGNTNYNPSIKS (SEQ ID NO: 2) | 2 |
| | CDR2 | | |
| | VH | CARAYGKLGFDY (SEQ ID NO: 3) | 3 |
| | CDR3 | | |
| | VH | QVQLKESGPGLVKPSQSLSLTCSVTGFSITTGGYWWTWIRQFPG | 19 |
| | | QKLEWMGYIFSSGNTNYNPSIKSRISITRDTSKNQFFLQLNSVTT | |
| | | EGDTARYYCARAYGKLGFDYWGQGTLVTVSS | |
| | VL | TLSSQHSTYTIG (SEQ ID NO: 4) | 4 |
| | CDR1 | | |
| | VL | VNSDGSHSKGD (SEQ ID NO: 5) | 5 |
| | CDR2 | | |
| | VL | GSSDSSGYV (SEQ ID NO: 6) | 6 |
| | CDR3 | | |
| | VL | QPVLTQSPSASASLGNSVKITCTLSSQHSTYTIGWYQQHPDKAP | 20 |
| | | KYVMYVNSDGSHSKGDGIPDRFSGSSSGAHRYLSISNIQPEDEAD | |
| | | YFCGSSDSSGYVFGSGTQLTVL | |
| GBM01-VH | | QVQLQESGPGLVKPSETLSLTCTVSGFSITTGGYWWTWIRQPPG | 48 |
| | | KGLEWIGYIFSSGNTNYNPSIKSRVTISVDTSKNQFSLKLSSVTAA | |
| | | DTAVYYCARAYGKLGFDYWGQGTLVTVSS | |
| GBM02-VH | | QVQLQESGPGLVKPSQTLSLTCTVSGFSITTGGYWWTWIRQHPG | 49 |
| | | KGLEWIGYIFSSGNTNYNPSIKSLVTISVDTSKNQFSLKLSSVTAA | |
| | | DTAVYYCARAYGKLGFDYWGQGTLVTVSS | |
| GBM03-VH | | QVQLQESGPGLVKPSQTLSLTCTVSGFSITTGGYWWTWIRQPPG | 50 |
| | | KGLEWIGYIFSSGNTNYNPSIKSRVTISVDTSKNQFSLKLSSVTAA | |
| | | DTAVYYCARAYGKLGFDYWGQGTLVTVSS | |
| GBM04-VH | | QVQLQESGPGLVKPSETLSLTCTVSGFSITTGGYWWTWIRQPPG | 51 |
| | | KGLEWIGYIFSSGNTNYNPSIKSRVTISRDTSKNQFSLKLSSVTAA | |
| | | DTAVYYCARAYGKLGFDYWGQGTLVTVSS | |
| GBM05-VH | | QVQLQESGPGLVKPSETLSLTCTVSGFSITTGGYWWTWIRQPPG | 52 |
| | | KGLEWIGYIFSSGNTNYNPSIKSRVTISVDTSKNQFSLKLSSVTAA | |
| | | DTARYYCARAYGKLGFDYWGQGTLVTVSS | |
| GBM06-VH | | QVQLQESGPGLVKPSETLSLTCTVSGFSITTGGYWWTWIRQPPG | 53 |
| | | KGLEWIGYIFSSGNTNYNPSIKSRISITRDTSKNQFFLQLNSVTTE | |
| | | GDTARYYCARAYGKLGFDYWGQGTLVTVSS | |
| GBM01-VL | | QLVLTQSPSASASLGASVKLTCTLSSQHSTYTIGWHQQQPEKGP | 54 |
| | | RYLMKVNSDGSHSKGDGIPDRFSGSSSGAERYLTISSLQSEDEAD | |
| | | YYCGSSDSSGYVFGSGTKVTVL | |
| GBM02-VL | | LPVLTQPPSASALLGASIKLTCTLSSQHSTYTIGWYQQRPGRSPQ | 55 |
| | | YIMKVNSDGSHSKGDGIPDRFMGSSSGADRYLTFSNLQSDDEAE | |
| | | YHCGSSDSSGYVFGSGTKVTVL | |
| Anti-CD83 ScFv 1 | | QVQLQESGPGLVKPSETLSLTCTVSGFSITTGGYWWTWIRQPPGK | 59 |
| | | GLEWIGYIFSSGNTNYNPSIKSRVTISVDTSKNQFSLKLSSVTAAD | |
| | | TAVYYCARAYGKLGFDYWGQGTLVTVSSGGGGSGGGGSGGGG | |
| | | SQLVLTQSPSASASLGASVKLTCTLSSQHSTYTIGWHQQQPEKGP | |
| | | RYLMKVNSDGSHSKGDGIPDRFSGSSSGAERYLTISSLQSEDEAD | |
| | | YYCGSSDSSGYVFGSGTKVTVL | |
| Anti-CD83 ScFv 2 | | QVQLQESGPGLVKPSQTLSLTCTVSGFSITTGGYWWTWIRQHPG | 60 |
| | | KGLEWIGYIFSSGNTNYNPSIKSLVTISVDTSKNQFSLKLSSVTAA | |
| | | DTAVYYCARAYGKLGFDYWGQGTLVTVSSGGGGSGGGGSGGG | |
| | | GSQLVLTQSPSASASLGASVKLTCTLSSQHSTYTIGWHQQQPEKG | |
| | | PRYLMKVNSDGSHSKGDGIPDRFSGSSSGAERYLTISSLQSEDEA | |
| | | DYYCGSSDSSGYVFGSGTKVTVL | |
| Anti-CD83 ScFv 3 | | QVQLQESGPGLVKPSQTLSLTCTVSGFSITTGGYWWTWIRQPPGK | 61 |
| | | GLEWIGYIFSSGNTNYNPSIKSRVTISVDTSKNQFSLKLSSVTAAD | |
| | | TAVYYCARAYGKLGFDYWGQGTLVTVSSGGGGSGGGGSGGGG | |
| | | SQLVLTQSPSASASLGASVKLTCTLSSQHSTYTIGWHQQQPEKGP | |
| | | RYLMKVNSDGSHSKGDGIPDRFSGSSSGAERYLTISSLQSEDEAD | |
| | | YYCGSSDSSGYVFGSGTKVTVL | |
| Anti-CD83 ScFv 4 | | QVQLQESGPGLVKPSETLSLTCTVSGFSITTGGYWWTWIRQPPGK | 62 |
| | | GLEWIGYIFSSGNTNYNPSIKSRVTISRDTSKNQFSLKLSSVTAAD | |
| | | TAVYYCARAYGKLGFDYWGQGTLVTVSSGGGGSGGGGSGGGG | |
| | | SQLVLTQSPSASASLGASVKLTCTLSSQHSTYTIGWHQQQPEKGP | |
| | | RYLMKVNSDGSHSKGDGIPDRFSGSSSGAERYLTISSLQSEDEAD | |
| | | YYCGSSDSSGYVFGSGTKVTVL | |
| Anti-CD83 ScFv 5 | | QVQLQESGPGLVKPSETLSLTCTVSGFSITTGGYWWTWIRQPPGK | 63 |
| | | GLEWIGYIFSSGNTNYNPSIKSRVTISVDTSKNQFSLKLSSVTAAD | |
| | | TARYYCARAYGKLGFDYWGQGTLVTVSSGGGGSGGGGSGGGG | |
| | | SQLVLTQSPSASASLGASVKLTCTLSSQHSTYTIGWHQQQPEKGP | |
| | | RYLMKVNSDGSHSKGDGIPDRFSGSSSGAERYLTISSLQSEDEAD | |
| | | YYCGSSDSSGYVFGSGTKVTVL | |
| Anti-CD83 ScFv 6 | | QVQLQESGPGLVKPSETLSLTCTVSGFSITTGGYWWTWIRQPPGK | 64 |
| | | GLEWIGYIFSSGNTNYNPSIKSRISITRDTSKNQFFLQLNSVTTEGD | |
| | | TARYYCARAYGKLGFDYWGQGTLVTVSSGGGGSGGGGSGGGG | |
| | | SQLVLTQSPSASASLGASVKLTCTLSSQHSTYTIGWHQQQPEKGP | |
| | | RYLMKVNSDGSHSKGDGIPDRFSGSSSGAERYLTISSLQSEDEAD | |
| | | YYCGSSDSSGYVFGSGTKVTVL | |
| Anti-CD83 ScFv 7 | | QVQLQESGPGLVKPSETLSLTCTVSGFSITTGGYWWTWIRQPPGK | 65 |
| | | GLEWIGYIFSSGNTNYNPSIKSRVTISVDTSKNQFSLKLSSVTAAD | |
| | | TAVYYCARAYGKLGFDYWGQGTLVTVSSGGGGSGGGGSGGGG | |
| | | SLPVLTQPPSASALLGASIKLTCTLSSQHSTYTIGWYQQRPGRSPQ | |
| | | YIMKVNSDGSHSKGDGIPDRFMGSSSGADRYLTFSNLQSDDEAE | |
| | | YHCGSSDSSGYVFGSGTKVTVL | |
| Anti-CD83 ScFv 8 | | QVQLQESGPGLVKPSQTLSLTCTVSGFSITTGGYWWTWIRQHPG | 66 |
| | | KGLEWIGYIFSSGNTNYNPSIKSLVTISVDTSKNQFSLKLSSVTAA | |
| | | DTAVYYCARAYGKLGFDYWGQGTLVTVSSGGGGSGGGGSGGG | |
| | | GSLPVLTQPPSASALLGASIKLTCTLSSQHSTYTIGWYQQRPGRSP | |
| | | QYIMKVNSDGSHSKGDGIPDRFMGSSSGADRYLTFSNLQSDDEA | |
| | | EYHCGSSDSSGYVFGSGTKVTVL | |
| Anti-CD83 ScFv 9 | | QVQLQESGPGLVKPSQTLSLTCTVSGFSITTGGYWWTWIRQPPGK | 67 |
| | | GLEWIGYIFSSGNTNYNPSIKSRVTISVDTSKNQFSLKLSSVTAAD | |
| | | TAVYYCARAYGKLGFDYWGQGTLVTVSSGGGGSGGGGSGGGG | |
| | | SLPVLTQPPSASALLGASIKLTCTLSSQHSTYTIGWYQQRPGRSPQ | |
| | | YIMKVNSDGSHSKGDGIPDRFMGSSSGADRYLTFSNLQSDDEAE | |
| | | YHCGSSDSSGYVFGSGTKVTVL | |
| Anti-CD83 ScFv 10 | | QVQLQESGPGLVKPSETLSLTCTVSGFSITTGGYWWTWIRQPPGK | 68 |
| | | GLEWIGYIFSSGNTNYNPSIKSRVTISRDTSKNQFSLKLSSVTAAD | |
| | | TAVYYCARAYGKLGFDYWGQGTLVTVSSGGGGSGGGGSGGGG | |
| | | SLPVLTQPPSASALLGASIKLTCTLSSQHSTYTIGWYQQRPGRSPQ | |
| | | YIMKVNSDGSHSKGDGIPDRFMGSSSGADRYLTFSNLQSDDEAE | |
| | | YHCGSSDSSGYVFGSGTKVTVL | |
| Anti-CD83 ScFv 11 | | QVQLQESGPGLVKPSETLSLTCTVSGFSITTGGYWWTWIRQPPGK | 68 |
| | | GLEWIGYIFSSGNTNYNPSIKSRVTISVDTSKNQFSLKLSSVTAAD | |
| | | TARYYCARAYGKLGFDYWGQGTLVTVSSGGGGSGGGGSGGGG | |
| | | SLPVLTQPPSASALLGASIKLTCTLSSQHSTYTIGWYQQRPGRSPQ | |
| | | YIMKVNSDGSHSKGDGIPDRFMGSSSGADRYLTFSNLQSDDEAE | |
| | | YHCGSSDSSGYVFGSGTKVTVL | |
| Anti-CD83 ScFv 12 | | QVQLQESGPGLVKPSETLSLTCTVSGFSITTGGYWWTWIRQPPGK | 70 |
| | | GLEWIGYIFSSGNTNYNPSIKSRISITRDTSKNQFFLQLNSVTTEGD | |
| | | TARYYCARAYGKLGFDYWGQGTLVTVSSGGGGSGGGGSGGGG | |
| | | SLPVLTQPPSASALLGASIKLTCTLSSQHSTYTIGWYQQRPGRSPQ | |
| | | YIMKVNSDGSHSKGDGIPDRFMGSSSGADRYLTFSNLQSDDEAE | |
| | | YHCGSSDSSGYVFGSGTKVTVL | |
| Anti-CD83 ScFv 13, | | QVQLKESGPGLVKPSQSLSLTCSVTGFSITTGGYWWTWIRQFPGQ | 71 |
| VH & VL derived | | KLEWMGYIFSSGNTNYNPSIKSRISITRDTSKNQFFLQLNSVTTEG | |
| from GMB00 | | DTARYYCARAYGKLGFDYWGQGTLVTVSSGGGGSGGGGSGGG | |
| | | GSQPVLTQSPSASASLGNSVKITCTLSSQHSTYTIGWYQQHPDKA | |
| | | PKYVMYVNSDGSHSKGDGIPDRFSGSSSGAHRYLSISNIQPEDEA | |
| | | DYFCGSSDSSGYVFGSGTQLTVL | |
| 96G08 | VH | SDGIS | 7 |
| | CDR1 | | |
| | VH | IISSGGNTYYASWAKG | 8 |
| | CDR2 | | |
| | VH | VVGGTYSI | 9 |
| | CDR3 | | |
| | VH | QCQSVEESGGRLVTPGTPLTLTCTVSGIDLSSDGISWVRQAPGKG | 72 |
| | | LEWIGIISSGGNTYYASWAKGRFTISRTSTTVDLKMTSLTTEDTA | |
| | | TYFCARVVGGTYSIWGQGTLVTVSS | |
| | H Chain | METGLRWLLLVAVLKGVQCQSVEESGGRLVTPGTPLTLTCTVSGID | 31 |
| | | LSSDGISWVRQAPGKGLEWIGIISSGGNTYYASWAKGRFTISRTST | |
| | | TVDLKMTSLTTEDTATYFCARVVGGTYSIWGQGTLVTVSSASTK | |
| | | GPSVYPLAPGSAAQTNSMVTLGCLVKGYFPEPVTVTWNSGSLSS | |
| | | GVHTFPAVLQSDLYTLSSSVTVPSSTWPSETVTCNVAHPASSTKV | |
| | | DKKIVPRDCGCKPCICTVPEVSSVFIFPPKPDVLTITLTPKVTCVVV | |
| | | DISKDDPEVQFSWFVDDVEVHTAQTQPREEQFNSTFRSVSELPIM | |
| | | HQDWLNGKEFKCRVNSAAFPAPIEKTISKTKGRPKAPQVYTIPPP | |
| | | KEQMAKDKVSLTCMITDFFPEDITVEWQWNGQPAENYKNTQPI | |
| | | MDTDGSYFVYSKLNVQKSNWEAGNTFTCSVLHEGLHNHHTEKS | |
| | | LSHSPGK | |
| | VL | QSSQSVYNNDFLS | 10 |
| | CDR1 | | |
| | VL | YASTLAS | 11 |
| | CDR2 | | |
| | VL | TGTYGNSAWYEDA | 12 |
| | CDR3 | | |
| | VL | QVLTQTASPVSAPVGGTVTINCQSSQSVYNNDFLSWYQQKPGQP | 73 |
| | | PKLLIYYASTLASGVPSRFKGSGSGTQFTLTISDLECDDAATYYC | |
| | | TGTYGNSAWYEDAFGGGTEVVVKRTP | |
| | L chain | MDTRAPTQLLGLLLLWLPGATFAQVLTQTASPVSAPVGGTVTIN | 32 |
| | | CQSSQSVYNNDFLSWYQQKPGQPPKLLIYYASTLASGVPSRFKGS | |
| | | GSGTQFTLTISDLECDDAATYYCTGTYGNSAWYEDAFGGGTEVV | |
| | | VKRTPVAPTVLLFPPSSAELATGTATIVCVANKYFPDGTVTWKV | |
| | | DGITQSSGINNSRTPQNSADCTYNLSSTLTLSSDEYNSHDEYTCQV | |
| | | AQDSGSPVVQSFSRKSC | |
| 95F04 | VH | SNAMI | 13 |
| | CDR1 | | |
| | VH | AMDSNSRTYYATWAKG | 14 |
| | CDR2 | | |
| | VH | GDGGSSDYTEM | 15 |
| | CDR3 | | |
| | VH | QCQSVEESGGRLVTPGTPLTLTCTVSGIDLSSNAMIWVRQAPREG | 74 |
| | | LEWIGAMDSNSRTYYATWAKGRFTISRTSSITVDLKITSPTTEDT | |
| | | ATYFCARGDGGSSDYTEMWGPGTLVTVSS | |
| | H Chain | METGLRWLLLVAVLKGVQCQSVEESGGRLVTPGTPLTLTCTVSGID | 33 |
| | | LSSNAMIWVRQAPREGLEWIGAMDSNSRTYYATWAKGRFTISRT | |
| | | SSITVDLKITSPTTEDTATYFCARGDGGSSDYTEMWGPGTLVTVS | |
| | | SASTKGPSVYPLAPGSAAQTNSMVTLGCLVKGYFPEPVTVTWNS | |
| | | GSLSSGVHTFPAVLQSDLYTLSSSVTVPSSTWPSETVTCNVAHPA | |
| | | SSTKVDKKIVPRDCGCKPCICTVPEVSSVFIFPPKPKDVLTITLTPK | |
| | | VTCVVVDISKDDPEVQFSWFVDDVEVHTAQTQPREEQFNSTFRS | |
| | | VSELPIMHQDWLNGKEFKCRVNSAAFPAPIEKTISKTKGRPKAPQ | |
| | | VYTIPPPKEQMAKDKVSLTCMITDFFPEDITVEWQWNGQPAENY | |
| | | KNTQPIMDTDGSYFVYSKLNVQKSNWEAGNTFTCSVLHEGLHN | |
| | | HHTEKSLSHSPGK | |
| | VL | QSSQSVYGNNELS | 16 |
| | CDR1 | | |
| | VL | QASSLAS | 17 |
| | CDR2 | | |
| | VL | LGEYSISADNH | 18 |
| | CDR3 | | |
| | VL | QAVVTQTTSPVSAPVGGTVTINCQSSQSVYGNNELSWYQQKPG | 75 |
| | | QPPKLLIYQASSLASGVPSRFKGSGSGTQFTLTISDLECDDAATYY | |
| | | CLGEYSISADNHFGGGTEVVVKRTP | |
| | L chain | MDTRAPTQLLGLLLLWLPGATFAQAVVTQTTSPVSAPVGGTVTINC | 34 |
| | | QSSQSVYGNNELSWYQQKPGQPPKLLIYQASSLASGVPSRFKGSG | |
| | | SGTQFTLTISDLECDDAATYYCLGEYSISADNHFGGGTEVVVKRT | |
| | | PVAPTVLLFPPSSAELATGTATIVCVANKYFPDGTVTWKVDGITQSSGI | |
| | | NNSRTPQNSADCTYNLSSTLTLSSDEYNSHDEYTCQVAQDSGSPVVQS | |
| | | FSRKSC | |
| 20D04 | H chain | METGLRWLLLVAVLKGVQCQSVEESGGRLVTPGTPLTLTCTVSGFS | 21 |
| | | LSNNAINWVRQAPGKGLEWIGYIWSGGLTYYANWAEGRFTISK | |
| | | TSTTVDLKMTSPTIEDTATYFCARGINNSALWGPGTLVTVSSGQP | |
| | | KAPSVFPLAPCCGDTPSSTVTLGCLVKGYLPEPVTVTWNSGTLTNGV | |
| | | RTFPSVRQSSGLYSLSSVVSVTSSSQPVTCNVAHPATNTKVDKTVAPST | |
| | | CSKPTCPPPELLGGPSVFIFPPKPKDTLMISRTPEVTCVVVDVSQDDP | |
| | | EVQFTWYINNEQVRTARPPLREQQFNSTIRVVSTLPIAHQDWLRGKEF | |
| | | KCKVHNKALPAPIEKTISKARGQPLEPKVYTMGPPREELSSRSVSLTC | |
| | | MINGFYPSDISVEWEKNGKAEDNYKTTPAVLDSDGSYFLYNKLSVPTS | |
| | | EWQRGDVFTCSVMHEALHNHYTQKSISRSPGK | |
| | L chain | MDMRAPTQLLGLLLLWLPGARCADVVMTQTPASVSAAVGGTVTI | 22 |
| | | NCQASESISNYLSWYQQKPGQPPKLLIYRTSTLASGVSSRFKGSG | |
| | | SGTEYTLTISGVQCDDVATYYCQCTSGGKFISDGAAFGGGTEVV | |
| | | VKGDPVAPTVLLFPPSSDEVATGTVTIVCVANKYFPDVTVTWEVDGT | |
| | | TQTTGIENSKTPQNSADCTYNLSSTLTLTSTQYNSHKEYTCKVTQGTTS | |
| | | VVQSFSRKNC | |
| 11G05 | H chain | METGLRWLLLVAVLKGVQCQSVEESGGRLVTPGTPLTLTCTVSGFT | 23 |
| | | ISDYDLSWVRQAPGEGLKYIGFIAIDGNPYYATWAKGRFTISKT | |
| | | STTVDLKITAPTTEDTATYFCARGAGDLWGPGTLVTVSSGQPKA | |
| | | PSVFPLAPCCGDTPSSTVTLGCLVKGYLPEPVTVTWNSGTLTNGVRTF | |
| | | PSVRQSSGLYSLSSVVSVTSSSQPVTCNVAHPATNTKVDKTVAPSTCSK | |
| | | PTCPPPELLGGPSVFIFPPKPKDTLMISRTPEVTCVVVDVSQDDPEVQ | |
| | | FTWYINNEQVRTARPPLREQQFNSTIRVVSTLPIAHQDWLRGKEFKC | |
| | | KVHNKALPAPIEKTISKARGQPLEPKVYTMGPPREELSSRSVSLTCMI | |
| | | NGFYPSDISVEWEKNGKAEDNYKTTPAVLDSDGSYFLYNKLSVPTSE | |
| | | WQRGDVFTCSVMHEALHNHYTQKSISRSPGK | |
| | L chain | MDTREPTQLLGLLLLWLPGARCADVVMTQTPASVSAAVGGTVTIN | 24 |
| | | CQSSKNVYNNNWLSWFQQKPGQPPKLLIYYASTLASGVPSRFR | |
| | | GSGSGTQFTLTISDVQCDDAATYYCAGDYSSSSDNGFGGGTEVV | |
| | | VKGDPVAPTVLLFPPSSDEVATGTVTIVCVANKYFPDVTVTWEVDGT | |
| | | TQTTGIENSKTPQNSADCTYNLSSTLTLTSTQYNSHKEYTCKVTQGTTS | |
| | | VVQSFSRKNC | |
| 14C12 | H chain | METGLRWLLLVAVLKGVHCQSVEESGGRLVTPGTPLTLTCTASGFS | 25 |
| | | RSSYDMSWVRQAPGKGLEWVGVISTAYNSHYASWAKGRFTISR | |
| | | TSTTVDLKMTSLTTEDTATYFCARGGSWLDLWGQGTLVTVSSG | |
| | | QPKAPSVFPLAPCCGDTPSSTVTLGCLVKGYLPEPVTVTWNSGTLTN | |
| | | GVRTFPSVRQSSGLYSLSSVVSVTSSSQPVTCNVAHPATNTKVDKTVAP | |
| | | STCSKPTCPPPELLGGPSVFIFPPKPKDTLMISRTPEVTCVVVDVSQD | |
| | | DPEVQFTWYINNEQVRTARPPLREQQFNSTIRVVSTLPIAHQDWLRG | |
| | | KEFKCKVHNKALPAPIEKTISKARGQPLEPKVYTMGPPREELSSRSVS | |
| | | LTCMINGFYPSDISVEWEKNGKAEDNYKTTPAVLDSDGSYFLYNKLSV | |
| | | PTSEWQRGDVFTCSVMHEALHNHYTQKSISRSPGK | |
| | L chain | MDXRAPTQLLGLLLLWLPGARCALVMTQTPASVSAAVGGTVTINC | 26 |
| | | QSSQSVYDNDELSWYQQKPGQPPKLLIYALASKLASGVPSRFKG | |
| | | SGSGTQFALTISGVQCDDAATYYCQATHYSSDWYLTFGGGTEV | |
| | | VVKGFPVAPTVLLFPPSSDEVATGTVTIVCVANKYFPDVTVTWEVDG | |
| | | TTQTTGTENSKTPQNSADCTYNLSSTLTLTSTQYNSHKEYTCKVTQGT | |
| | | TSVVQSFSRKNC | |
| 020B08 | H chain | METGLRWLLLVAVLKGVQCQSVEESGGRLVTPGTPLTLTCTVSGFS | 27 |
| | | LSSYDMTWVRQAPGKGLEWIGIIYASGTTYYANWAKGRFTISK | |
| | | TSTTVDLKVTSPTIGDTATYFCAREGAGVSMTLWGPGTLVTVSS | |
| | | GQPKAPSVFPLAPCCGDTPSSTVTLGCLVKGYLPEPVTVTWNSGTLT | |
| | | NGVRTFPSVRQSSGLYSLSSVVSVTSSSQPVTCNVAHPATNTKVDKTVA | |
| | | PSTCSKPTCPPPELLGGPSVFIFPPKPKDTLMISRTPEVTCVVVDVSQ | |
| | | DDPEVQFTWYINNEQVRTARPPLREQQFNSTIRVVSTLPIAHQDWLR | |
| | | GKEFKCKVHNKALPAPIEKTISKARGQPLEPKVYTMGPPREELSSRSV | |
| | | SLTCMINGFYPSDISVEWEKNGKAEDNYKTTPAVLDSDGSYFLYNKLS | |
| | | VPTSEWQRGDVFTCSVMHEALHNHYTQKSISRSPGK | |
| | L chain | MDMRAPTQLLGLLLLWLPGARCAYDMTQTPASVEVAVGGTVTIK | 28 |
| | | CQASQSISTYLDWYQQKPGQPPKLLIYDASDLASGVPSRFKGSG | |
| | | SGTQFTLTISDLECADAATYYCQQGYTHSNVDNVFGGGTEVVV | |
| | | KGDPVAPTVLLFPPSSDEVATGTVTIVCVANKYFPDVTVTWEVDGTT | |
| | | QTTGIENSKTPQNSADCTYNLSSTLTLTSTQYNSHKEYTCKVTQGTTSV | |
| | | VQSFSRKNC | |
| 006G05 | H chain | METGLRWLLLVAVLKGVQCQSVEESGGRLVSPGTPLTLTCTASGFS | 29 |
| | | LSSYDMSWVRQAPGKGLEYIGIISSSGSTYYASWAKGRFTISKTS | |
| | | TTVDLEVTSLTTEDTATYFCSREHAGYSGDTGHLWGPGTLVTV | |
| | | SSGQPKAPSVFPLAPCCGDTPSSTVTLGCLVKGYLPEPVTVTWNSGT | |
| | | LTNGVRTFPSVRQSSGLYSLSSVVSVTSSSQPVTCNVAHPATNTKVDKT | |
| | | VAPSTCSKPTCPPPELLGGPSVGIGPPKPKDTLMISRTPEVTCVVVDV | |
| | | SQDDPEVQFTWYINNEQVRTARPPLREQQFNSTIRVVSTLPIAHQDW | |
| | | LRGKEFKCKVHNKALPAPIEKTISKARGQPLEPKVYTMGPPREELSSR | |
| | | SVSLTCMINGFYPSDISVEWEKNGKAEDNYKTTPAVLDSDGSYFLYNK | |
| | | LSVPTSEWQRGDVFTCSVMHEALHNHYTQKSISRSPGK | |
| | L chain | MDMRAPTQLLGLLLLWLPGARCAYDMTQTPASVEVAVGGTVAIK | 30 |
| | | CQASQSVSSYLAWYQQKPGQPPKPLIYEASMLAAGVSSRFKGSG | |
| | | SGTDFTLTISDLECDDAATYYCQQGYSISDIDNAFGGGTEVVVK | |
| | | GDPVAPTVLLFPPSSDEVATGTVTIVCVANKYFPDVTVTWEVDGTTQT | |
| | | TGIENSKTPQNSADCTYNLSSTLTLTSTQYNSHKEYTCKVTQGTTSVVQ | |
| | | SFSRKNC | |
| Linker | | GGGGSGGGGSGGGGS | 56 |
| Anti- | VH-CH1 | QVQLVQSGGAVVQPGRSLRLSCAASGFTFSTYGMHWVRQAPGK | 35 |
| CD83 | | GLEWVAAVSYDGSNKYYADFVKGRFTISRDNPKNTLYLQMNSL | |
| Clone | | RADDTAVYYCARRGGLDIWGQGTTVTVSSASTKGPSVFPLAPSSK | |
| A* | | STSGGTAALGCLVKDYFPEPVTVSWNSGALTSGVHTFPAVLQSSGLYS | |
| | | LSSVVTVPSSSLGTQTYICNVNHKPSNTKVDKKVEPKSCAAA | |
| | VL1 | LTQPPPASGTPGQQRVTISCSGSSSNIGSNTVNWYQQLPGTAPKL | 36 |
| | | LIYYGNDQRPSGVPDRFSASKSGTSASLAISGLQSEDEAHYYCAA | |
| | | WDGSLNGGVIFGGGTKVTLG (SEQ ID NO: 36) | |
| | VL 2 | VTQPPSASGTPGQRVTISCSGSSSNIGTNPVNWYQQLPGTAPKLLI | 37 |
| | | YTTDQRPSGVPDRFSGSKSGTSASLAISGLQSEDEADYYCAAWD | |
| | | DSLSGLYVFGTGTKVTVLG (SEQ ID NO: 37) | |
| | VL 3 | MTHTPLSLSVTPGQPASISCKSSQSLLHSDGKTYLYWYLQRPGQ | 38 |
| | | SPQPLIYEVSNRFSGVPDRFSGSGSGTDFTLKISRVQAEDVGVYY | |
| | | CMQSLQLWTFGQGTKVEIKR (SEQ ID NO: 38) | |
| | VL 4 | MTQSPLSLPVTLGQPASISCRSSQSLIHSDGNTYLDWFQQRPGQS | 39 |
| | | PRRLIYKVSNRDSGVPDRFSGSGSGTDFTLRISRVEAEDIGVYYC | |
| | | MQATHWPRTFGQGTKVEIKR(SEQ ID NO: 39) | |
| | VL 5 | MTQSPLSLPVTLGQPASISCRSSQSLVDSAGNTFLHWFHQRPGQS | 40 |
| | | PRRLIYKVSNRDSGVPDRFSGSGSGTDFTLKISRVEAEDVGVYYC | |
| | | MQGTHWPRTFGQGTKVEIKR(SEQ ID NO: 40) | |
| | VL 6 | LTQSPLSLPVTLGQPASISCKSSQSLVDSDGNTYLNWFQQRPGQS | 41 |
| | | PRRLIYKVSNRDSGVPDRFSGSGSGTDFTLKISRVEAEDVGVYYC | |
| | | MQGTHWPRTFGQGTKVEIKR(SEQ ID NO: 41) | |
| | VL 7 | MTQSPLSLPVTLGQPASISCRSSQSLVHSDGNMYLNWFQQRPGQ | 42 |
| | | SPRRLIYKVSNRDSGVPDRFSGSGSGTDFTLKISRVEAEDVGVYY | |
| | | CMQATQPTWTFGQGTKLEIKR(SEQ ID NO: 42) | |
| | VL 8 | MTQSPSSLSASVGDRVTITCQASQDISNYLNWYQQKPGKAPKLLI | 43 |
| | | YDASNLETGVPSRFSGSGSGTDFTFTISSATYYCQQTYQGTKLEIK | |
| | | R (SEQ ID NO: 43) | |
| | VL 9 | MTQSPSSLSASVGHPVTITCRASQSLISYLNWYHQKPGKAPKLLI | 44 |
| | | YAASILQSGVPSRFSGSGSGTDFTLTISSLQPENFASYYCQHTDSF | |
| | | PRTFGHGTKVEIKR (SEQ ID NO: 44). | |
| | VL 10 | LTQPPSASGTPGQGVTISCRGSTSNIGNNVVNWYQHVPGSAPKL | 45 |
| | | LIWSNIQRPSGIPDRFSGSKSGTSASLAISGLQSEDQAVYYCAVW | |
| | | DDGLAGWVFGGGTTVTVLS (SEQ ID NO: 45) | |
| | VL 11 | MTQAPVVSVALEQTVRITCQGDSLAIYYDFWYQHKPGQAPVLVI | 46 |
| | | YGKNNRPSGIPHRFSGSSSNTDSLTITGAQAEDEADYYCNSRDSS | |
| | | GNHWVFGGGTNLTVLG (SEQ ID NO: 46) | |
| | VL 12 | LTQSPLSLPVTLGQPASISCKSNQSLVHSDGNTYLNWFQQRPGQ | 47 |
| | | SPRRLIYKVSNRDSGVPDRFSGSGSGTDFTLKINRVEAEDVGVYY | |
| | | CMQGTQWPRTFGGQGTKLDIKR(SEQ ID NO: 47) | |

| | | |
|---|---|---|
| ScFv | QPVLTQSPSASASLGNSVKITCTLSSQHSTYTIGWYQQHPDKAPK | 57 |
| | YVMYVNSDGSHSKGDGIPDRFSGSSSGAHRYLSISNIQPEDEADY | |
| | FCGSSDSSGYVFGSGTQLTVLRAAASSGGGGSGGGGSGGGGSQP | |
| | VLTQSPSASASLGNSVKITCTLSSQHSTYTIGWYQQHPDKAPKYV | |
| | MYVNSDGSHSKGDGIPDRFSGSSSGAHRYLSISNIQPEDEADYFC | |
| | GSSDSSGYVFGSGTQLTVLRAAA (SEQ ID NO: 57) | |
| ScFv | QVQLKESGPGLVKPSQSLSLTCSVTGFSITTGGYWWTWIRQFPGQ | 58 |
| | KLEWMGYIFSSGNTNYNPSIKSRISITRDTSKNQFFLQLNSVTTEG | |
| | DTARYYCARAYGKLGFDYWGQGTLVTVSSGGGGSGGGGSGGG | |
| | GSQVQLKESGPGLVKPSQSLSLTCSVTGFSITTGGYWWTWIRQFP | |
| | GQKLEWMGYIFSSGNTNYNPSIKSRISITRDTSKNQFFLQLNSVTT | |
| | EGDTARYYCARAYGKLGFDYWGQGTLVTV (SEQ ID NO: 58) | |

Signal peptide: N-terminal italicized region
CDRs: underlined and in boldface
Constant region: C-terminal italicized region
*Antibody (see US 20170335006, the relevant disclosures are incorporated by reference for
the subject matter and purpose referenced herein)

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A bi-specific genetically modified immune cell, comprising a first antigen binding moiety that is specific to CD83 and a second antigen binding moiety that is specific to interleukin 6 receptor (IL-6R).

2. The bi-specific genetically modified immune cell of claim 1, wherein the immune cell expresses a first chimeric antigen receptor (CAR) that comprises the first antigen binding moiety, and a second chimeric antigen receptor (CAR) that comprises the second antigen binding moiety, and wherein the first CAR further comprises a first co-stimulatory signaling domain and a first intracellular signaling domain and the second CAR further comprises a second co-stimulatory signaling domain and a second intracellular signaling domain.

3. The bi-specific genetically modified immune cell of claim 1, wherein the immune cell expresses a bi-specific chimeric antigen receptor (CAR), which comprises the first antigen binding moiety, the second antigen binding moiety, a co-stimulatory signaling domain, and an intracellular signaling domain.

4. The bi-specific genetically modified immune cell of claim 1, wherein the first antigen binding moiety specific to CD83 is a single chain variable fragment (scFv) that binds CD83.

5. The bi-specific genetically modified immune cell of claim 4, wherein the scFv that binds CD83 comprises the same heavy chain complementary determining regions as a reference anti-CD83 antibody and/or the same light chain complementary determining regions as the reference anti-CD83 antibody, and wherein the reference anti-CD83 antibody is GMB00, Clone Clone 11G05, Clone 14C12, Clone 020B08, Clone 006G05, Clone 96G08, or Clone 95F04; optionally wherein the reference anti-CD83 antibody is GMB00, 96G08, or 95F04.

6. The bi-specific genetically modified immune cell of claim 4, wherein the scFv that binds CD83 comprises (a) the same heavy chain variable region (VH) as GMB01, GMB02, GMB03, GMB04, GMB05, or GMB06, and/or the same light chain variable region (VL) as GMB01, or GMB02; or (b) the same VH and/or the same VL as the reference antibody Clone Clone 11G05, Clone 14C12, Clone 020B08, Clone 006G05, Clone 96G08, or Clone 95F04.

7. The bi-specific genetically modified immune cell of claim 6, wherein the scFv that binds CD83 comprises an amino acid sequence selected from the group consisting of SEQ. ID. Nos.: 59-71.

8. The bi-specific genetically modified immune cell of claim 1, wherein the second antigen binding moiety specific to IL-6R is a single chain variable fragment (scFv) that binds the IL-6R or an IL-6R ligand.

9. The bi-specific genetically modified immune cell of claim 1, wherein the second antigen binding moiety binds IL-6Rα or GP130.

10. The bi-specific genetically modified immune cell of claim 1, wherein the second antigen binding moiety specific to IL-6R is a single chain variable fragment (scFv) derived from Tocilizumab, Sarilumab, monoclonal antibody clones TZLS-501, ALX-0061, and BCD-089.

11. The bi-specific genetically modified immune cell of claim 2, wherein the first co-stimulatory signaling, the second co-stimulatory domain, and/or the co-stimulatory domain in the bispecific CAR is a co-stimulatory signaling domain of CD27, CD28, 4-1BB, OX40, CD30, CD40, PD-1, ICOS, lymphocyte function-associated antigen-1 (LFA-1), CD2, CD7, LIGHT, NKG2C, or B7-H3, optionally wherein the first co-stimulatory signaling domain, the second co-stimulatory signaling domain, and/or the co-stimulatory signaling domain in the bispecific CAR is a CD28 co-stimulatory signaling domain or a 4-1BB co-stimulatory signaling domain.

12. The bi-specific genetically modified immune cell of claim 2, wherein the first intracellular signaling, the second intracellular signaling domain, and/or the intracellular signaling domain in the bispecific CAR is a CD3ζ signaling domain.
Thbasset87!

13. The bi-specific genetically modified immune cell of claim 2, wherein the first CAR, the second CAR, or the bispecific CAR further comprises a hinge domain, a transmembrane domain, or a combination thereof.

14. The bi-specific genetically modified immune cell of claim 1, wherein the immune cell is a T cell.

15. A bi-specific chimeric antigen receptor (CAR), wherein the bi-specific CAR comprises a first antigen binding moiety specific to CD83 and a second antigen binding moiety specific to IL-6R.

16. The bi-specific CAR of claim 15, wherein the first antigen binding moiety specific to CD83 is a single chain variable fragment (scFv) that binds CD83.

17. The bi-specific CAR of claim 15, wherein the second antigen binding moiety specific to IL-6R is a single chain variable fragment (scFv) that binds the IL-6R or an IL-6R ligand.

18. The bi-specific CAR of claim 15, which further comprises a co-stimulatory signaling domain and an intracellular signaling domain, optionally wherein the co-stimulatory signaling domain is a co-stimulatory signaling domain of CD27, CD28, 4-1BB, OX40, CD30, CD40, PD-1, ICOS, lymphocyte function-associated antigen-1 (LFA-1), CD2, CD7, LIGHT, NKG2C, or B7-H3, and optionally wherein the intracellular signaling domain is a CD3ζ signaling domain.

19. The bi-specific CAR of claim 15, which further comprises a hinge domain, a transmembrane domain, or a combination thereof.

20. A method for suppressing alloreactive immune responses in a subject, the method comprising administering to a subject in need thereof an effective amount of the bi-specific genetically modified immune cells set forth in claim 1, thereby suppressing alloreactive donor cells in the subject.

21. The method of claim 20, wherein the donor cells are bone marrow cells comprising alloreactive T-cells, dendritic cells, or a combination thereof.

22. The method of claim 20, wherein the subject has undergone or is undergoing a therapy comprising a checkpoint inhibitor, which optionally is an anti-PD-1 antibody, an anti-PD-L1 antibody, or an anti-CTLA-4 antibody.

23. The method of claim 20, wherein the subject is a human patient in need of transplantation of the alloreactive donor cells, optionally is at risk of GVHD or rejection by the alloreactive cells.

24. The method of claim 20, wherein the bi-specific genetically modified immune cells suppress alloreactive cells in the allogenic donor cells but preserve donor immunity against a target antigen, which optionally is a cancer antigen and/or an antigen from an infectious pathogen.


SEQUENCE LISTING (75 sequences)

SEQ ID NO: 1 - protein, 12 aa, Artificial Sequence
  Synthetic
GFSITTGGYWWT

SEQ ID NO: 2 - protein, 17 aa, Artificial Sequence
  Synthetic
GYIFSSGNTNYNPSIKS

SEQ ID NO: 3 - protein, 12 aa, Artificial Sequence
  Synthetic
CARAYGKLGFDY

SEQ ID NO: 4 - protein, 12 aa, Artificial Sequence
  Synthetic
TLSSQHSTYTIG

SEQ ID NO: 5 - protein, 11 aa, Artificial Sequence
  Synthetic
VNSDGSHSKGD

SEQ ID NO: 6 - protein, 9 aa, Artificial Sequence
  Synthetic
GSSDSSGYV

SEQ ID NO: 7 - protein, 5 aa, Artificial Sequence
  Synthetic
SDGIS

SEQ ID NO: 8 - protein, 16 aa, Artificial Sequence
  Synthetic
IISSGGNTYYASWAKG

SEQ ID NO: 9 - protein, 8 aa, Artificial Sequence
  Synthetic
VVGGTYSI

SEQ ID NO: 10 - protein, 13 aa, Artificial Sequence
  Synthetic
QSSQSVYNNDFLS

SEQ ID NO: 11 - protein, 7 aa, Artificial Sequence
  Synthetic
YASTLAS

SEQ ID NO: 12 - protein, 13 aa, Artificial Sequence
  Synthetic
TGTYGNSAWYEDA

SEQ ID NO: 13 - protein, 5 aa, Artificial Sequence
  Synthetic
SNAMI

SEQ ID NO: 14 - protein, 16 aa, Artificial Sequence
  Synthetic
AMDSNSRTYYATWAKG

SEQ ID NO: 15 - protein, 11 aa, Artificial Sequence
  Synthetic
GDGGSSDYTEM

SEQ ID NO: 16 - protein, 13 aa, Artificial Sequence
  Synthetic
QSSQSVYGNNELS

SEQ ID NO: 17 - protein, 7 aa, Artificial Sequence
  Synthetic
QASSLAS

SEQ ID NO: 18 - protein, 11 aa, Artificial Sequence
  Synthetic
LGEYSISADNH

SEQ ID NO: 19 - protein, 120 aa, Artificial Sequence
  Synthetic
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSS

SEQ ID NO: 20 - protein, 111 aa, Artificial Sequence
  Synthetic
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 21 - protein, 454 aa, Artificial Sequence
  Synthetic
METGLRWLLLVAVLKGVQCQSVEESGGRLVTPGTPLTLTCTVSGFSLSNNAINWVRQAPGKGLEWIGYIWSGGLTYYANWAEGRFTISKTSTTVDLKMTSPTIEDTATYFCARGINNSALWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 22 - protein, 239 aa, Artificial Sequence
  Synthetic
MDMRAPTQLLGLLLLWLPGARCADVVMTQTPASVSAAVGGTVTINCQASESISNYLSWYQQKPGQPPKLLIYRTSTLASGVSSRFKGSGSGTEYTLTISGVQCDDVATYYCQCTSGGKFISDGAAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 23 - protein, 452 aa, Artificial Sequence
  Synthetic
METGLRWLLLVAVLKGVQCQSVEESGGRLVTPGTPLTLTCTVSGFTISDYDLSWVRQAPGEGLKYIGFIAIDGNPYYATWAKGRFTISKTSTTVDLKITAPTTEDTATYFCARGAGDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 24 - protein, 238 aa, Artificial Sequence
  Synthetic
MDTREPTQLLGLLLLWLPGARCADVVMTQTPASVSAAVGGTVTINCQSSKNVYNNNWLSWFQQKPGQPPKLLIYYASTLASGVPSRFRGSGSGTQFTLTISDVQCDDAATYYCAGDYSSSSDNGFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 25 - protein, 454 aa, Artificial Sequence
  Synthetic
METGLRWLLLVAVLKGVHCQSVEESGGRLVTPGTPLTLTCTASGFSRSSYDMSWVRQAPGKGLEWVGVISTAYNSHYASWAKGRFTISRTSTTVDLKMTSLTTEDTATYFCARGGSWLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 26 - protein, 239 aa, Artificial Sequence
  Synthetic
  misc_feature (3)..(3): Xaa can be any naturally occurring amino acid
MDXRAPTQLLGLLLLWLPGARCALVMTQTPASVSAAVGGTVTINCQSSQSVYDNDELSWYQQKPGQPPKLLIYALASKLASGVPSRFKGSGSGTQFALTISGVQCDDAATYYCQATHYSSDWYLTFGGGTEVVVKGFPVAPTVLLFPPSSDEVATGTVTIVCVANKYFPDVTVTWEVDGTTQTTGTENSKTPQNSADCTYNLSSTLTLTSTQYNSHKEYTCKVTQGTTSVVQSFSRKNC

SEQ ID NO: 27 - protein, 456 aa, Artificial Sequence
  Synthetic
METGLRWLLLVAVLKGVQCQSVEESGGRLVTPGTPLTLTCTVSGFSLSSYDMTWVRQAPGKGLEWIGIIYASGTTYYANWAKGRFTISKTSTTVDLKVTSPTIGDTATYFCAREGAGVSMTLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 28 - protein, 236 aa, Artificial Sequence
  Synthetic
MDMRAPTQLLGLLLLWLPGARCAYDMTQTPASVEVAVGGTVTIKCQASQSISTYLDWYQQKPGQPPKLLIYDASDLASGVPSRFKGSGSGTQFTLTISDLECADAATYYCQQGYTHSNVDNVFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 29 - protein, 459 aa, Artificial Sequence
  Synthetic
METGLRWLLLVAVLKGVQCQSVEESGGRLVSPGTPLTLTCTASGFSLSSYDMSWVRQAPGKGLEYIGIISSSGSTYYASWAKGRFTISKTSTTVDLEVTSLTTEDTATYFCSREHAGYSGDTGHLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVGIGPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYNKLSVPTSEWQRGDVFTCSVMHEALHNHYTQKSISRSPGK

SEQ ID NO: 30 - protein, 236 aa, Artificial Sequence
  Synthetic
MDMRAPTQLLGLLLLWLPGARCAYDMTQTPASVEVAVGGTVAIKCQASQSVSSYLAWYQQKPGQPPKPLIYEASMLAAGVSSRFKGSGSGTDFTLTISDLECDDAATYYCQQGYSISDIDNAFGGGTEVVVKGDPVAPTVLLFPPSSDEVATGTVTIVCVANKYFPDVTVTWEVDGTTQTTGIENSKTPQNSADCTYNLSSTLTLTSTQYNSHKEYTCKVTQGTTSVVQSFSRKNC

SEQ ID NO: 31 - protein, 455 aa, Artificial Sequence
  Synthetic
METGLRWLLLVAVLKGVQCQSVEESGGRLVTPGTPLTLTCTVSGIDLSSDGISWVRQAPGKGLEWIGIISSGGNTYYASWAKGRFTISRTSTTVDLKMTSLTTEDTATYFCARVVGGTYSIWGQGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 240 aa, Artificial Sequence
  Synthetic
MDTRAPTQLLGLLLLWLPGATFAQVLTQTASPVSAPVGGTVTINCQSSQSVYNNDFLSWYQQKPGQPPKLLIYYASTLASGVPSRFKGSGSGTQFTLTISDLECDDAATYYCTGTYGNSAWYEDAFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 33 - protein, 460 aa, Artificial Sequence
  Synthetic
METGLRWLLLVAVLKGVQCQSVEESGGRLVTPGTPLTLTCTVSGIDLSSNAMIWVRQAPREGLEWIGAMDSNSRTYYATWAKGRFTISRTSSITVDLKITSPTTEDTATYFCARGDGGSSDYTEMWGPGTLVTVSSASTKGPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 239 aa, Artificial Sequence
  Synthetic
MDTRAPTQLLGLLLLWLPGATFAQAVVTQTTSPVSAPVGGTVTINCQSSQSVYGNNELSWYQQKPGQPPKLLIYQASSLASGVPSRFKGSGSGTQFTLTISDLECDDAATYYCLGEYSISADNHFGGGTEVVVKRTPVAPTVLLFPPSSAELATGTATIVCVANKYFPDGTVTWKVDGITQSSGINNSRTPQNSADCTYNLSSTLTLSSDEYNSHDEYTCQVAQDSGSPVVQSFSRKSC

SEQ ID NO: 35 - protein, 221 aa, Artificial Sequence
  Synthetic
QVQLVQSGGAVVQPGRSLRLSCAASGFTFSTYGMHWVRQAPGKGLEWVAAVSYDGSNKYYADFVKGRFTISRDNPKNTLYLQMNSLRADDTAVYYCARRGGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAA

SEQ ID NO: 36 - protein, 110 aa, Artificial Sequence
  Synthetic
LTQPPPASGTPGQQRVTISCSGSSSNIGSNTVNWYQQLPGTAPKLLIYYGNDQRPSGVPDRFSASKSGTSASLAISGLQSEDEAHYYCAAWDGSLNGGVIFGGGTKVTLG

SEQ ID NO: 37 - protein, 109 aa, Artificial Sequence
  Synthetic
VTQPPSASGTPGQRVTISCSGSSSNIGTNPVNWYQQLPGTAPKLLIYTTDQRPSGVPDRFSGSKSGTSASLAISGLQSEDEADYYCAAWDDSLSGLYVFGTGTKVTVLG

SEQ ID NO: 38 - protein, 109 aa, Artificial Sequence
  Synthetic
MTHTPLSLSVTPGQPASISCKSSQSLLHSDGKTYLYWYLQRPGQSPQPLIYEVSNRFSGVPDRFSGSGSGTDFTLKISRVQAEDVGVYYCMQSLQLWTFGQGTKVEIKR

SEQ ID NO: 39 - protein, 110 aa, Artificial Sequence
  Synthetic
MTQSPLSLPVTLGQPASISCRSSQSLIHSDGNTYLDWFQQRPGQSPRRLIYKVSNRDSGVPDRFSGSGSGTDFTLRISRVEAEDIGVYYCMQATHWPRTFGQGTKVEIKR

SEQ ID NO: 40 - protein, 110 aa, Artificial Sequence
  Synthetic
MTQSPLSLPVTLGQPASISCRSSQSLVDSAGNTFLHWFHQRPGQSPRRLIYKVSNRDSGVPDRFSGSGSGTDFTLKISRVEAEDVGVYYCMQGTHWPRTFGQGTKVEIKR

SEQ ID NO: 41 - protein, 110 aa, Artificial Sequence
  Synthetic
LTQSPLSLPVTLGQPASISCKSSQSLVDSDGNTYLNWFQQRPGQSPRRLIYKVSNRDSGVPDRFSGSGSGTDFTLKISRVEAEDVGVYYCMQGTHWPRTFGQGTKVEIKR

SEQ ID NO: 42 - protein, 110 aa, Artificial Sequence
  Synthetic
MTQSPLSLPVTLGQPASISCRSSQSLVHSDGNMYLNWFQQRPGQSPRRLIYKVSNRDSGVPDRFSGSGSGTDFTLKISRVEAEDVGVYYCMQATQPTWTFGQGTKLEIKR

SEQ ID NO: 43 - protein, 92 aa, Artificial Sequence
  Synthetic
MTQSPSSLSASVGDRVTITCQASQDISNYLNWYQQKPGKAPKLLIYDASNLETGVPSRFSGSGSGTDFTFTISSATYYCQQTYQGTKLEIKR

SEQ ID NO: 44 - protein, 105 aa, Artificial Sequence
  Synthetic
MTQSPSSLSASVGHPVTITCRASQSLISYLNWYHQKPGKAPKLLIYAASILQSGVPSRFSGSGSGTDFTLTISSLQPENFASYYCQHTDSFPRTFGHGTKVEIKR

SEQ ID NO: 45 - protein, 108 aa, Artificial Sequence
  Synthetic
LTQPPSASGTPGQGVTISCRGSTSNIGNNVVNWYQHVPGSAPKLLIWSNIQRPSGIPDRFSGSKSGTSASLAISGLQSEDQAVYYCAVWDDGLAGWVFGGGTTVTVLS

SEQ ID NO: 46 - protein, 105 aa, Artificial Sequence
  Synthetic
MTQAPVVSVALEQTVRITCQGDSLAIYYDFWYQHKPGQAPVLVIYGKNNRPSGIPHRFSGSSSNTDSLTITGAQAEDEADYYCNSRDSSGNHWVFGGGTNLTVLG

SEQ ID NO: 47 - protein, 111 aa, Artificial Sequence
  Synthetic
LTQSPLSLPVTLGQPASISCKSNQSLVHSDGNTYLNWFQQRPGQSPRRLIYKVSNRDSGVPDRFSGSGSGTDFTLKINRVEAEDVGVYYCMQGTQWPRTFGGQGTKLDIKR

SEQ ID NO: 48 - protein, 119 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 49 - protein, 119 aa, Artificial Sequence
  Synthetic
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSS

SEQ ID NO: 50 - protein, 119 aa, Artificial Sequence
  Synthetic
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSS

SEQ ID NO: 51 - protein, 119 aa, Artificial Sequence
  Synthetic
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSS

SEQ ID NO: 52 - protein, 119 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSS

SEQ ID NO: 53 - protein, 120 aa, Artificial Sequence
  Synthetic
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSS

SEQ ID NO: 54 - protein, 111 aa, Artificial Sequence
  Synthetic
QLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 55 - protein, 111 aa, Artificial Sequence
  Synthetic
LPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 56 - protein, 15 aa, Artificial Sequence
  Synthetic
GGGGSGGGGSGGGGS

SEQ ID NO: 57 - protein, 247 aa, Artificial Sequence
  Synthetic
QPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAASSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVLRAAA

SEQ ID NO: 58 - protein, 253 aa, Artificial Sequence
  Synthetic
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTV

SEQ ID NO: 59 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 60 - protein, 245 aa, Artificial Sequence
  Synthetic
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 61 - protein, 245 aa, Artificial Sequence
  Synthetic
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 62 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 63 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 64 - protein, 246 aa, Artificial Sequence
  Synthetic
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSQLVLTQSPSASASLGASVKLTCTLSSQHSTYTIGWHQQQPEKGPRYLMKVNSDGSHSKGDGIPDRFSGSSSGAERYLTISSLQSEDEADYYCGSSDSSGYVFGSGTKVTVL

SEQ ID NO: 65 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 66 - protein, 245 aa, Artificial Sequence
  Synthetic
QVQLQESGPGLVKPSQTLSLTCTVSGFSITTGGYWWTWIRQHPGKGLEWIGYIFSSGNTNYNPSIKSLVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 67 - protein, 245 aa, Artificial Sequence
  Synthetic
QVQLQESGPGLVKPSQTLSLTCTVSGFSITTGGYWWTWIRQPPGKGLEWIGYIFSSGNTNYNPSIKSRVTISVDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 68 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISRDTSKNQFSLKLSSVTAADTAVYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 69 - protein, 245 aa, Artificial Sequence
  Synthetic
QVQLQESGPGLVKPSETLSLTCTVSGFSITTGGYWWTWIRQPPGKGLEWIGYIFSSGNTNYNPSIKSRVTISVDTSKNQFSLKLSSVTAADTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 70 - protein, 246 aa, Artificial Sequence
  Synthetic
QVQLQESGPGLVKPSETLSLTCTVSGFSITTGGYWWTWIRQPPGKGLEWIGYIFSSGNTNYNPSIKSRISITRDTSKNQFFLQLNSVTTEGDTARYYCARAYGKLGFDYWGQGTLVTVSSGGGGSGGGGSGGGGSLPVLTQPPSASALLGASIKLTCTLSSQHSTYTIGWYQQRPGRSPQYIMKVNSDGSHSKGDGIPDRFMGSSSGADRYLTFSNLQSDDEAEYHCGSSDSSGYVFGSGTKVTVL

SEQ ID NO: 71 - protein, 246 aa, Artificial Sequence
  Synthetic
QVQLKESGPGLVKPSQSLSLTCSVTGFSITTGGYWWTWIRQFPGQKLEWMGYIFSSGNTNYNPSIKSRISITRDTSKNQFFLQLNSVTTEGDTARYYCARAYGKLGFDYWGQGTLVTVSSGGGGSGGGGSGGGGSQPVLTQSPSASASLGNSVKITCTLSSQHSTYTIGWYQQHPDKAPKYVMYVNSDGSHSKGDGIPDRFSGSSSGAHRYLSISNIQPEDEADYFCGSSDSSGYVFGSGTQLTVL

SEQ ID NO: 72 - protein, 115 aa, Artificial Sequence
  Synthetic
QCQSVEESGGRLVTPGTPLTLTCTVSGIDLSSDGISWVRQAPGKGLEWIGIISSGGNTYYASWAKGRFTISRTSTTVDLKMTSLTTEDTATYFCARVVGGTYSIWGQGTLVTVSS

SEQ ID NO: 73 - protein, 115 aa, Artificial Sequence
  Synthetic
QVLTQTASPVSAPVGGTVTINCQSSQSVYNNDFLSWYQQKPGQPPKLLIYYASTLASGVPSRFKGSGSGTQFTLTISDLECDDAATYYCTGTYGNSAWYEDAFGGGTEVVVKRTP

SEQ ID NO: 74 - protein, 119 aa, Artificial Sequence
  Synthetic
QCQSVEESGGRLVTPGTPLTLTCTVSGIDLSSNAMIWVRQAPREGLEWIGAMDSNSRTYYATWAKGRFTISRTSSITVDLKITSPTTEDTATYFCARGDGGSSDYTEMWGPGTLVTVSS

SEQ ID NO: 75 - protein, 114 aa, Artificial Sequence
  Synthetic
QAVVTQTTSPVSAPVGGTVTINCQSSQSVYGNNELSWYQQKPGQPPKLLIYQASSLASGVPSRFKGSGSGTQFTLTISDLECDDAATYYCLGEYSISADNHFGGGTEVVVKRTP